(19) United States
(12) Patent Application Publication
(10) Pub. No.: US 2026/0071188 A1
(43) Pub. Date: Mar. 12, 2026

(54) METHODS OF INDUCING CELLULAR QUIESCENCE

(71) Applicant: The Regents of the University of California, Oakland, CA (US)

(72) Inventors: Jernej Murn, Riverside, CA (US); Sihem Cheloufi, Riverside, CA (US)

(21) Appl. No.: 19/325,024

(22) Filed: Sep. 10, 2025

Related U.S. Application Data
(60) Provisional application No. 63/694,113, filed on Sep. 12, 2024.

(51) Int. Cl. C12N 5/071

(52) U.S. Cl. CPC C12N 5/0686; C12N 2501/30; C12N 2501/727

(57) ABSTRACT

The present disclosure provides methods for inducing cellular quiescence—defined as a reversible arrest of cell proliferation—by inhibiting ribosome biogenesis. The methods involve administering a quiescence-inducing agent, such as a small molecule inhibitor, peptide, antisense oligonucleotide, or targeted degrader, that interferes with rRNA transcription, processing, ribosomal protein synthesis, or ribosome assembly. The agent may be applied to cultured cells, tissues, organs, or organoids, across a range of cell types and species. In some embodiments, the methods reduce metabolic activity and enhance stress resistance in non-dividing or post-mitotic cells. By slowing proliferation and/or metabolism while preserving viability, these methods enable improved preservation of cells, tissues, and organs-enhancing transplantation logistics, biobanking, regenerative medicine workflows, and biological transport. Additional applications include studies of cell cycle regulation, tissue regeneration, stress biology, aging, and induction of organismal hypometabolism (e.g., torpor or hibernation). The disclosure includes examples demonstrating quiescence induction using chemical biology systems, pharmacological agents, and genetic approaches.

### INCORPORATION BY REFERENCE TO ANY PRIORITY APPLICATIONS

Any and all applications for which a foreign or domestic priority claim is identified, for example, in the Application Data Sheet as filed with the present application are hereby incorporated by reference under 37 CFR 1.57, and Rules 4.18 and 20.6, including U.S. Provisional Application No. 63/694,113 filed Sep. 12, 2024, which is hereby incorporated by reference in its entirety.

### STATEMENT REGARDING FEDERALLY SPONSORED R&D

This invention was made with government support under Grant Number 2054195 awarded by the National Science Foundation. The government has certain rights in the invention.

### RELATED FIELD

Aspects of the present disclosure relate generally to the field of cell proliferation and metabolism. In some embodiments, compositions and methods are provided for inducing reversible cell quiescence.

### BACKGROUND

In a nutritive environment, proliferating cells invest most of their energy into growth and division. However, under nutrient deprivation, single-celled organisms enter a state of quiescence, a state of reversible proliferative arrest characterized by reduced metabolic activity that protects cells from stress and secures their survival until nutrients again become available. Cellular quiescence is also commonly used by multi-celled organisms, not only for survival of their individual cells, but for the benefit of the entire organism, including its development, long-term reproductive capacity, and tissue repair. In addition, cellular quiescence plays essential roles in control of body size, aging and longevity, and is dysregulated in a range of human pathologies, such as autoimmune disorders and cancer. Despite the importance of quiescence in all domains of life, little is still known about the molecular pathways that induce cells to enter quiescence.

While cellular quiescence is itself a primitive biological process, it is thought that quiescence has diversified over the course of evolution to serve many distinct biological needs. The expectation is thus that the cellular pathways underlying quiescence are conserved, but that they respond to various regulatory inputs in single-celled organisms and in metazoans. Indeed, the variety of quiescence-inducing signals (ranging from nutrient limitation in bacteria or yeast to altered availability of growth factors and cytokines in metazoans), entry points (largely from G1 but occasionally from other phases of the cell cycle), and durations (from a few days to several decades), suggests a diversity of regulatory schemes. However, the key to understanding and manipulating cellular quiescence is definition of the common underlying mechanisms.

### BRIEF SUMMARY

The following provides a brief summary of certain aspects of the disclosure, but does not constitute a comprehensive summary of all aspects.

Disclosed herein are methods for inducing quiescence in various cell types, such as, but not limited to, mammalian cells, including mouse and human cells. Presented are methods that in living cells inhibit ribosome biogenesis, which maintains cellular viability and reversibly arrests cellular proliferation that may be accompanied by reduction in cellular metabolism, including but not limited to reduced rate of DNA synthesis, reduced ATP production, reduced rate of gene transcription, and reduced rate of protein biosynthesis by cells.

In some embodiments, the induced cellular quiescence is achieved by genetic, chemical biology-based, pharmacological, peptide-based, or antibody-based approaches to inhibit the action or reduce the expression of cellular molecules that are involved in ribosome biogenesis, including biosynthesis of ribosomal RNA (rRNA). The said cellular molecules include but are not limited to RNA polymerase I, which is responsible for transcription of the precursor rRNA (pre-rRNA), and molecules, including enzymes, that participate in post-transcriptional (downstream) processing of pre-rRNA. In some embodiments, the quiescence-inducing genetic agent, chemical, peptide or protein, is added in the appropriate form and concentration directly to cells, including but not limited to cells grown in culture in their full growth media. Alternatively, the quiescence-inducing genetic agent, chemical, peptide or protein can be added to the solution used for incubation, preservation, or perfusion of a tissue or organ.

In some embodiments, the induced cellular quiescence can be maintained for various periods of time from 2 days to 14 days, or longer (e.g., from 2 days to 5 days, or from 3 days to 7 days, or from 3 days to 14 days, or longer than 14 days) by continued incubation of cells with the quiescence-inducing genetic agent, chemical compound, peptide or protein. In some embodiments, the induced cellular quiescence can be released and cells allowed to re-enter proliferation by removal of the genetic agent, chemical, peptide or protein used for the induction and maintenance of quiescence via washing with media, physiologic solution, or other appropriate means.

The methods of the invention can be used for a variety of purposes in basic biomedical research, including studies of cell differentiation and organismal development, aging and longevity, stress tolerance and survival, tissue regeneration, wound healing, fertility, immune responses, and other biological processes where cellular quiescence plays a critical role. This includes states where dysregulated cellular quiescence contributes to organismal disorders, including muscular, neurological, autoimmune disorders, hypo- and hyperplasias, and cancer. Furthermore, these methods are useful for biotechnological, clinical, therapeutic, or exploratory purposes, such as preservation of cells, tissues, and organs for transplantations or biobanking, therapy of the said organismal disorders, slowing down of organismal aging, and induction of hibernation, stasis, or torpor of organisms.

Even short-term extension of viability for transplantable biomaterials-on the scale of days-could have a transformative impact on clinical logistics and therapeutic outcomes. By enabling temporary quiescence in cells, tissues, or organs, the invention allows preservation in a dormant yet viable state, reducing metabolic demand while maintaining recovery potential. This added time could support broader donor-recipient matching, global distribution of donor organs, immunological testing (e.g., HLA compatibility and immune tolerance), and elective transplant scheduling. Accordingly, the invention is well suited for applications in transplantation medicine, organ banking, and emergency preparedness, where increased shelf life translates directly into increased access and success rates.

Non-limiting examples of the present disclosure are also encapsulated by the following enumerated alternatives:

1. A method of inducing cellular quiescence, the method comprising:
(a) incubating a plurality of cells with a quiescence-inducing agent in an amount sufficient to inhibit ribosome biogenesis and induce cellular quiescence;
(b) optionally maintaining the quiescent state by continued incubation with the quiescence-inducing agent; and
(c) optionally releasing the plurality of cells from quiescence by removing the quiescence-inducing agent.

2. The method of alternative 1, wherein the quiescence-inducing agent inhibits RNA polymerase I.

3. The method of any one of the previous alternatives, wherein the quiescence-inducing agent is CX-5461 or a derivative thereof.

4. The method of any one of the previous alternatives, wherein the quiescence-inducing agent is a heterobifunctional small molecule that induces degradation of a ribosome biogenesis protein via the ubiquitin-proteasome system.

5. The method of alternative 4, wherein the quiescence-inducing agent induces targeted degradation through a degradation tag, and wherein the degradation tag is FKBP12^{F36V} and the heterobifunctional molecule is dTAG-13 or dTAG^{v}-1.

6. The method of any one of the previous alternatives, wherein the quiescence-inducing agent comprises an auxin-inducible degron system and auxin or a derivative thereof.

7. The method of any one of the previous alternatives, wherein the quiescence-inducing agent inhibits expression of a gene involved in ribosome biogenesis via RNA interference, antisense oligonucleotides, or a CRISPR-based method.

8. The method of alternative 7, wherein the quiescence-inducing agent is an antisense oligonucleotide that binds pre-rRNA and blocks cleavage by an rRNA-processing endonuclease.

9. The method of any one of the previous alternatives, wherein the plurality of cells comprises a tissue, organ, or organoid.

10. The method of any one of the previous alternatives, further comprising lowering the temperature of the plurality of cells to below 35° C., freezing, or vitrifying the plurality of cells during or after induction of quiescence.

11. The method of any one of the previous alternatives, wherein the quiescence-inducing agent is administered in vitro, ex vivo, or in situ.

12. The method of any one of the previous alternatives, wherein the quiescent state is maintained for a period of 2 to 14 days.

13. The method of any one of the previous alternatives, wherein releasing the plurality of cells from quiescence comprises washing the cells or omitting further administration of the quiescence-inducing agent.

14. The method of any one of the previous alternatives, wherein the plurality of cells are human and/or mammalian.

15. A method of reducing metabolic activity in non-dividing cells, the method comprising administering to the cells quiescence-inducing agent, thereby decreasing energy consumption while maintaining cell viability.

16. A method of treating a subject in need thereof, the method comprising administering to the subject a quiescence-inducing agent.

17. The method of alternative 16, wherein the quiescence-inducing agent prolongs the anti-aging and/or longevity of the subject.

18. The method of alternative 16 or 17, wherein the subject has a disease or disorder.

19. The method of alternative 18, wherein the disease or disorder is a metabolic disease or disorder.

20. A method of cell, tissue, organ, or organism preservation, the method comprising administering to the cell, tissue organ, or organism a quiescence-inducing agent.

21. The method of alternative 20, wherein the method further comprises cryopreserving the cell, tissue, organ, or organism.

22. The method of alternative 20 or 21, wherein the cell, tissue, or organ is later part of a cell, tissue, or organ transfer.

23. The method of any one of alternatives 20-22, wherein the method further comprises storing the cell, tissue, organ, or organism for at least one day, week, month, or year.

24. The method of any one of alternatives 20-23, wherein the method further comprises inactivating and/or removing the quiescence-inducing agent.

25. The method of alternative 24, wherein once the quiescence-inducing agent is inactive and/or removed, proliferation is resumed.

26. The method of alternative 24 or 25, further comprising administering an proliferation-inducing agent.

27. The method of alternative 26, wherein the proliferation-induced agent is a nutrient and/or a nutrient-rich broth.

28. The method of any one of alternatives 20-27, wherein the cell, tissue, or organ is mammalian and/or human.

29. The method of any one of alternatives 20-27, wherein the organism is a lab animal, optionally wherein the organism is a fly, mouse, worm, or fish.

30. The method of any one of alternatives 20-28, wherein the cell, tissue, or organ comprises a heart, liver, lung, skin, kidney, egg, sperm, fertilized egg, pancreas, or intestinal cell, or any combination thereof.

31. The method of any one of the previous alternatives, wherein the quiescence-inducing agent has activity in preventing ribosomal and/or rRNA biogenesis.

32. The method of any one of the previous alternatives, wherein the quiescence-inducing agent promotes the degradation of a ribosome and/or an rRNA.

33. The method of any one of the previous alternatives, wherein the quiescence-inducing agent targets RNA polymerase I, RNase MRP, LASIL, or any combination thereof.

34. The method of alternative 33, wherein the targeting inhibits activity and/or reduces the total copy number of RNA polymerase I, RNase MRP, and/or LASIL present in a cell, tissue, organ, organ system, and/or organism.

35. A method of inducing quiescence in a cell, tissue, organ, organoid, organ system, tumor, organism, or any combination thereof, the method comprising:
administering to the cell, tissue, organ, organoid, tumor, organ system, or
organism an effective concentration of a quiescence-inducing agent,
wherein the effective concentration of the quiescence-inducing agent is one in which the total number of active ribosomes is reduced in the cell, tissue, organ, organoid, tumor, organ system, or organism compared to an untreated cell, tissue, organ, organoid, tumor, or organism.

36. The method of any one of the previous alternatives, wherein the quiescence-inducing agent inhibits ribosomal biogenesis and/or rRNA biogenesis.

37. The method of any one of the previous alternatives, wherein the quiescence-inducing agent inhibits activity, expression, and/or the total number of an at least one ribosome biogenesis protein and/or rRNA biogenesis protein present in a cell, tissue, organ, organoid, organ system, tumor, and/or organism.

38. The method of any one of the previous alternatives, wherein the at least one ribosome biogenesis protein and/or rRNA biogenesis protein is involved in at least 1, 2, 3, 4, or 5 of: rRNA transcription, rRNA processing, rRNA modification, ribosomal protein formation, transport of assembled ribosomal subunits, or any combination thereof.

39. The method of any one of the previous alternatives, wherein the quiescence-inducing agent inhibits activity and/or reduces the total copy number of RNA polymerase I, RNase MRP, LASIL, or any combination thereof.

40. The method of any one of the previous alternatives, wherein the quiescence-inducing agent is CX-5461 or a derivative thereof.

41. The method of any one of the previous alternatives, wherein the quiescence-inducing agent induces targeted degradation of the ribosome biogenesis protein and/or rRNA biogenesis protein.

42. The method of alternative 41, wherein the quiescence-inducing agent induces targeted degradation through a degradation tag, and wherein the degradation tag is FKBP12^{F36V}.

43. The method of any one of the previous alternatives, wherein the quiescence-inducing agent comprises an auxin-inducible degron system and auxin or a derivative thereof.

44. The method of any one of the previous alternatives, wherein the quiescence-inducing agent inhibits expression of a gene involved in ribosome biogenesis via RNA interference, an antisense oligonucleotide, or a CRISPR-based method.

45. The method of any one of the previous alternatives, wherein total number of active ribosomes in the cell, tissue, organ, organoid, tumor, or organism is reduced by at least about 10%, 20%, 30%, 40%, 50%, 60%, 70%, 80%, 90%, 95%, 99%, 100%, or any number between about 10% and about 100%, compared to an analogous cell, tissue, organ, organoid, tumor, or organism that is not administered the quiescence-inducing agent.

46. The method of any one of the previous alternatives, the method further comprising lowering the temperature of the plurality of cells to below 35° C., freezing, or vitrifying the plurality of cells during or after induction of quiescence.

47. The method of any one of the previous alternatives, wherein the quiescence-inducing agent is administered in vitro, ex vivo, or in situ.

48. The method of any one of the previous alternatives, wherein the quiescent state is maintained for a period of at least about 2, 4, 6, 8, 10, 12, or 14 days.

49. The method of any one of the previous alternatives, the method further comprising inhibiting and/or removing the quiescence-inducing agent, wherein the cell, tissue, organ, organoid, tumor, or organism is released from quiescence.

50. The method of alternative 49, wherein inhibiting and/or removing the quiescence-inducing agent comprises washing the cells and/or omitting further administration of the quiescence-inducing agent.

51. The method of any one of the previous alternatives, further comprising contacting the cell, tissue, organ, organoid, tumor, or organism with a nutrient-enriched medium to promote recovery from quiescence.

52. A method of reducing metabolic activity in a non-dividing cell, tissue, organ, organoid, tumor, or organism, the method comprising administering a quiescence-inducing agent, thereby decreasing energy consumption while maintaining viability.

53. A method of promoting the viability and/or shelf-life of a cell, tissue, organ, organoid, tumor, or organism, the method comprising administering a quiescence-inducing agent.

54. The method of alternative 53, further comprising cryopreserving the cell, tissue, organ, organoid, tumor, organ system, or organism.

### Clarification of Scope

The present application includes specific examples of pharmacological and peptide-based agents that can be used to induce cellular quiescence by targeting ribosome biogenesis. However, this disclosure does not claim novel composition-of-matter inventions such as new chemical entities, structurally modified peptides, or delivery formulations-except those explicitly described herein. Any future development of proprietary compounds, optimized biologics, or targeted delivery systems is expected to fall outside the scope of this filing and may be the subject of separate intellectual property filings.

### BRIEF DESCRIPTION OF THE DRAWINGS

FIG. 1. depicts a nonlimiting example cartoon schematic of key regulators of rRNA biogenesis. Pol I and several key endonucleases (red) involved in early 47S pre-rRNA (blue) processing are highlighted.

FIG. 2. depicts a nonlimiting example cartoon schematic illustrating the dTAG strategy for rapid and inducible depletion of RPP40, a protein subunit of RNase MRP. BSDR, blasticidin resistance gene; L, linker; C, addback codons.

FIGS. 3A-3B depict nonlimiting examples of the inducible depletion of RPP40 in human cells. FIG. 3A depicts an immunoblot analysis of depletion of the endogenous (tagged) RPP40 using anti-HA antibody in HEK293T (C40) cells treated with DMSO or dTAG for the indicated time periods. Actin serves as a loading control (n=5). FIG. 3B depicts microscopy images of the endogenous RPP40 detected by immunofluorescence using anti-HA antibody in HEK293T (C40) cells treated with DMSO or dTAG for 3 h. DAPI was used to visualize nuclei. Scale bar, 20 μm.

FIGS. 4A-4D depict nonlimiting examples of the inducible depletion of RNase MRP in human cells. FIG. 4A depicts an example northern blot analysis of RMRP from HEK293T (C40) cells (n=3) treated with DMSO or dTAG for 3 h or 24 h. SNORD3A (U3 snoRNA) serves as a loading control. FIG. 4B depicts an example of the relative quantification by qPCR of RNA samples as in FIG. 4A (n=3). Data are shown as mean±SD. *, p<0.001 (Student's t test). FIG. 4C depicts a nonlimiting example of RNA FISH imaging of RMRP in HEK293T (C40) cells treated with DMSO or dTAG for 3 h. Scale bar, 20 μm. FIG. 4D depicts an example immunoblot analysis of RNase MRP protein subunits in lysates of HEK293T (C40) cells treated with DMSO or dTAG for 24 h (n=3).

FIGS. 5A-5B depict nonlimiting example northern and immunofluorescence analysis of rRNA precursors. FIG. 5A depicts a nonlimiting example cartoon schematic indicating where the northern probes mid (green, 5′ of the RNase MRP cleavage site) and 3′ end (purple, 3′ of the MRP cleavage site), align within ITS1 of pre-rRNA and the structure of each RNA species (left panel). Orange diagrams and asterisks (structure unknown) indicate noncanonical rRNA precursors. Location of the annotated RNase MRP cleavage site within ITS1 is also shown. This schematic is generated from the information provided via northern blot (right panel). FIG. 5B depicts nonlimiting representative images of HEK293T (C40) cells treated with DMSO or dTAG for 24 h h and stained for DNA content (blue) and the ITS1 segment of rRNA (green). Scale bar, 50 μm.

FIGS. 6A-6D depict nonlimiting examples of the rapid depletion of RNase MRP induces cellular quiescence. FIG. 6A depicts a nonlimiting example quantification of the growth of HEK293T (C40) cells over the indicated periods of time. Cells were treated either continuously with DMSO (black; control) or with dTAG for 3 days (brown), 7 days (red), or 14 days (pink) and then dTAG was washed out (WO). Color-coded arrows indicate the day of WO. Dashed pink line indicates cell viability during the 14-day treatment time course. Data are shown as mean±SD (n>=3). FIG. 6B depicts nonlimiting example representative microscopy brightfield views of C40 cells on the indicated days of treatment with either DMSO or dTAG, as indicated. Scale bar, 20 μm. FIG. 6C depicts nonlimiting example quantifications for the viability of dTAG-treated C40 cells analyzed by the Apoptosis Kit (ThermoFisher, Cat. nr. A35136). Cells were treated with dTAG for the indicated periods of time or with puromycin at 2 μg/ml for 12 h (Puro), co-stained with annexin V to detect externalized phosphatidylserine in apoptotic cells and SYTOX™ AADvanced™ stain to detect dead cells, and analyzed by flow cytometry. Proportions of viable, apoptotic, and dead cells were determined according to the manufacturer's instructions. FIG. 6D depicts a nonlimiting example correlation between the day of dTAG washout and the time period after the washout until the first significant increase in cell number (blue) or until the mean cell number increased by 10-fold (red).

FIGS. 7A-7B depict nonlimiting examples for the depletion and restoration of RNase MRP subunits. FIG. 7A depicts a nonlimiting example time-course immunoblot analysis of RNase MRP protein subunits in HEK293T (C40) cells treated with dTAG for 3 days before dTAG washout (n=3). FIG. 7B depicts a nonlimiting example quantification for the expression changes of RMRP (right) evaluated by qPCR of RNA isolated from HEK293T (C40) cells treated with dTAG for 3 days (n=3). Data are shown as mean±SD. *, p<0.05; **, p<0.01 (Student's t test).

FIGS. 8A-8C depict nonlimiting examples of restored RNase MRP levels and resumed cellular proliferation rely on genetic targeting of RPP40. FIG. 8A depicts a nonlimiting example immunoblot analysis of RNase MRP protein subunits, including the endogenously tagged RPP40 (HA-RPP40) and ectopic RPP40 (Flag-RPP40), in lysates of Dox-inducible HEK293T (C40) cells treated with DMSO or dTAG with or without Dox, as indicated (n=3). FIG. 8B depicts a nonlimiting example relative quantification of RMRP by qPCR of RNA isolated from Dox-inducible HEK293T (C40) cells treated with DMSO or dTAG with or without Dox, as indicated (n=3). Data are shown as mean±SD. *, p<0.05; **, p=0.0005 (Student's t test); ns, not significant. FIG. 8C depicts a nonlimiting example quantification of growth of Dox-inducible HEK293T (C40) cells treated either continuously with DMSO (black; control) or continuously with dTAG and in the presence of Dox from day 7 onward (orange; arrow indicates the time of Dox addition). Dashed orange line indicates viability of the dTAG and Dox-treated cells. Data are shown as mean±SD (n=3).

FIGS. 9A-8B depict nonlimiting examples for the quiescence of colorectal carcinoma cells HCT116. FIG. 9A depicts a nonlimiting example immunoblot analysis using an anti-HA antibody of lysates of HCT116 cells engineered for inducible degradation of endogenous RPP40, treated with DMSO or dTAG for 24 h (n=3). FIG. 9B depicts a nonlimiting example quantification for the growth of HCT116 cells engineered for inducible degradation of the endogenous RPP40. Cells were treated either continuously with DMSO (black; control), with dTAG for 3 days (red; red arrow indicates day 3 when dTAG was washed out, D3 WO), or continuously with dTAG (dark red, dTAG (on)). Dashed red line indicates viability of cells treated with dTAG for 3 days. Data are shown as mean±SD (n=3).

FIGS. 10A-10C depicts nonlimiting examples for cell cycle analysis of dTAG-treated HEK293T (C40) cells by flow cytometry. FIG. 10A depicts a nonlimiting example of cells treated with DMSO or dTAG for the indicated times, pulse-labeled with EdU for 1 h and stained for the incorporated EdU (with Alexa Fluor 594, AF594) and DNA (with FxCycle Violet Stain), then analyzed by flow cytometry. In each dot plot, matching DMSO-treated control cells are shown in gray. FIG. 10B depicts a nonlimiting example quantification of the relative mean fluorescence intensity (MFI) calculated by subtracting the mean EdU-AF594 intensity of unstained cells from the mean EdU-AF594 intensity of stained cells in Mid-S phase at the indicated times of DMSO or dTAG treatment. FIG. 10C depicts a nonlimiting example quantification of the phases of the cell cycle quantified as indicated by the gating in FIG. 10A. Active indicates cells synthesizing DNA and static cells not synthesizing DNA.

FIGS. 11A-11B depict nonlimiting examples for the decline in global translation in quiescent cells. FIG. 11A depicts a nonlimiting example puromycin incorporation assay. HEK293T (C40) cells treated with dTAG, as indicated, were incubated with puromycin for 30 min. Cycloheximide (CHX) treatment was performed 10 min prior to puromycin and served as a negative control. Puromycilated proteins in cell lysates were detected by immunoblotting using an anti-puromycin antibody (n=3). FIG. 11B depicts a nonlimiting example quantification for puromycin-incorporated protein levels in FIG. 11A; quantified and normalized by ACTIN levels. Data are shown as mean±SD. *, p<0.01; **, p<0.0005 (Student's t test); ns, not significant.

FIGS. 12A-12C depict nonlimiting example for the reduced metabolic activity in quiescent cells. FIG. 12A depicts a nonlimiting example energetic map quantifying ATP production via glycolysis and via oxidative phosphorylation (OXPHOS), showing the reduced ATP production rate by dTAG-treated HEK293T (C40) cells. Control or dTAG-treated cells, as indicated, were analyzed by the ATP rate assay using Seahorse analyzer (Agilent). ATP production rates through glycolysis or oxidative phosphorylation were determined. Data are shown as mean±SD (n=6). FIG. 12B depicts a nonlimiting example quantification showing the decreased mitochondrial activity of dTAG-treated HEK293T (C40) cells, assessed by MitoTracker staining (n=2). unst, unstained. FIG. 12C depicts a nonlimiting example quantification of mitochondrial DNA (mtDNA), showing the decreased mtDNA content of dTAG-treated HEK293T (C40) cells relative to controls. Measured by qPCR and normalized to genomic DNA. Data are shown as mean±SD. *, p=0.0178; **, p<0.01 (Student's t test).

FIGS. 13A-13B depict nonlimiting examples of the reduced overall transcriptional activity and decline in other critical cellular functions in quiescent cells. FIG. 13A depicts a nonlimiting example quantification of the rate of RNA synthesis in dTAG-treated HEK293T (C40) cells, measured by pulse-labeling with 5-ethynyluridine (EU) followed by flow cytometry. Actinomycin D (ActD)-treated cells served as a transcriptionally repressed control (n=3). FIG. 13B depicts a nonlimiting example quantification for the percent of normalized signaling, comparing changes in key cellular functions during transition of HEK293T (C40) cells from proliferation to quiescence. Time courses of average signals normalized to 100% for DMSO-treated cells are shown.

FIGS. 14A-14B depict nonlimiting examples of the induction of cellular quiescence using a Pol I inhibitor in HEK293T cells. FIG. 14A depicts a nonlimiting cartoon structure of CX-5461, an inhibitor of RNA polymerase I. FIG. 14B depicts a nonlimiting example quantification showing the growth of wild-type HEK293T cells over the indicated periods of time. Cells were treated either continuously with vehicle (phosphate buffer; gray, control), with 2 μM CX-5462 for 4 days and then washed out (WO; light green), or continuously with CX-5461 (dark green, (on)). Dotted lines indicate cell viability. Data are shown as mean±SD (n=3).

FIGS. 15A-15B depict nonlimiting examples of the induction of cellular quiescence using a Pol I inhibitor in mouse NIH/3T3 fibroblasts. FIG. 15A depicts a nonlimiting example quantification for the growth of wild-type NIH/3T3 cells over the indicated periods of time. Cells were treated either continuously with vehicle (phosphate buffer; blue, control) or were treated with 2 μM CX-5462 for 5 days prior to wash out of the compound (D5 WO; orange arrow). FIG. 15B depicts a nonlimiting example quantification for cell viability during the time course shown in FIG. 15A. Data are shown as mean±SD (n>=3).

FIGS. 16A-16C depict nonlimiting examples for the targeted degradation of the largest Pol I protein subunit, RPA194, induces quiescence in HCT116 cells. FIG. 16A depicts a nonlimiting example time-course immunoblot analysis of RPA194 in AID degron-edited HCT116 cells treated with doxycycline for 2 days (Dox only), Dox and auxin for 2 or 6 days (AUX 2D and AUX 6D), or with Dox and AUX for 6 days prior to washout for 4, 6, or 8 days (WO 10D, WO 12D, or WO 14D, respectively) (n=3). FIG. 16B depicts a nonlimiting example quantification for the growth of RPA194-AID degron-edited HCT116 cells over the indicated periods of time. Cells were treated either continuously with Dox (blue; control), or with Dox and AUX for 3 or 6 days prior to washout (red and dark red), or continuously with Dox and AUX (orange). FIG. 16C depicts a nonlimiting example quantification for cell viability during the time course shown in FIG. 16B. Data are shown as mean±SD (n>=3).

FIGS. 17A-17B depicts nonlimiting examples for the induction of cellular quiescence via targeted degradation of LASIL. FIG. 17A depicts a nonlimiting example immunoblot analysis of depletion of the endogenous LASIL as well as ectopic LASIL fused in-frame with KFBP12^{F36V} using anti-LASIL antibody in HEK293T cells treated with DMSO or dTAG for 1 h. Actin serves as a loading control (n=3). FIG. 17B depicts a nonlimiting example quantification for the growth of the edited HEK293T cells over the indicated periods of time. Cells were treated either continuously with vehicle (DMSO; blue, control) or with dTAG for 5 days prior to washout of dTAG at 5 days (D5 WO; red). Data are shown as mean±SD (n>=3).

### DETAILED DESCRIPTION

Aspects of the present disclosure relate to compositions and methods for inducing quiescence in various cells, tissues, organs, organoids, tumors, organ systems, or organisms. In some embodiments, the cells, tissues, organs, organoids, tumors, organ systems, and/or organisms are mammalian. In some embodiments, the cells, tissues, organs, organoids, tumors, organ systems, or organisms are mouse or human. In some embodiments, metabolism of the cells, tissues, organs, organoids, tumors, or organisms is altered during or after induction of quiescence. In some embodiments, there is a reduced rate of DNA synthesis, reduced ATP production, reduced rate of gene transcription, reduced rate of protein biosynthesis, or any combination thereof, associated with induction or maintenance of quiescence is achieved. In some embodiments, the quiescence is reversible.

A unifying feature of quiescent cells is their remarkably low overall level of protein biosynthesis, as low as 1% or less compared to proliferating cells. This is notable because translation, including protein biosynthesis and ribosome biogenesis, is by far the largest consumer of energy during cell proliferation as well as a key driver of diverse cellular functions and growth. A major reduction in the rate of translation can thus save a starving cell a substantial amount of energy to secure its survival. In fact, studies in bacteria and yeast demonstrate a universal engagement of active mechanisms to attenuate translation when nutrients become limiting. It remains unknown whether repression of energetically the most consuming processes, including ribosome biogenesis, itself serves as a strategy for induction of cellular quiescence.

In some embodiments, the method of the present disclosure comprises inhibiting ribosome biogenesis. In some embodiments, the synthesis of ribosomal subunits is inhibited. In some embodiments, the synthesis and/or processing of rRNA is inhibited. In some embodiments, rRNA biogenesis is inhibited. In some embodiments, the polymerization of ribosomal subunits is inhibited. In some embodiments, ribosome assembly and formation of the final ribosomal complex are inhibited. In some embodiments, ribosomal proteins themselves are targeted (for example, by degradation). In some embodiments, the transport of intermediate ribosomal subunits from the nucleolus to the nucleoplasm and/or from the nucleus to the cytoplasm is inhibited. In some embodiments, the inhibition of ribosome biogenesis is reversible.

Although aspects of the present disclosure relate to specific methods of inhibiting ribosome subunit synthesis and/or the formation of the final ribosomal complex, it will be understood that any method that similarly targets ribosomal biogenesis is predicted to be effective. Non-limiting examples of such methodology include gene-targeting approaches (for example, CRISPR-based gene editing), small molecule inhibitors of ribosome biogenesis factors, small molecule inhibitors of ribosomal complex formation, and methods for targeting ribosomal proteins or other ribosome biogenesis factors for degradation.

Non-Limiting Example of Molecular Targets for Arresting Ribosome Biogenesis and/or Inducing Quiescence

In some embodiments, the quiescence-inducing agent modulates at least 1, 2, 3, 4, 5, 6, 7, 8, 9, or 10 factor(s) involved in ribosome biogenesis. For example, in some embodiments the quiescence-inducing agent increases, decreases, reduces, prevents, or eliminates one or more of: the activity, stability, expression, or trafficking of factors required for ribosome biogenesis, or any combination thereof. Non-limiting classes and examples of genes, proteins, and molecular factors involved in ribosome biogenesis that can be targeted by a quiescent-inducing agent include:

1. Factors required for rRNA transcription, such as POLRIA, POLRIB, POLRIC, ROLRID, POLRIE, POLRIF, POLRIG, POLRIH, RRN3/TIF-IA, SLI complex, UBTF, and any combination thereof.

2. Factors required for pre-rRNA processing, such as RNase MRP, LASIL, UTP23, UTP24, NOB1, RCL1, EXOSC10, and any combination thereof.

3. Factors required for rRNA modification, such as FBL (fibrillarin), DKC1 (dyskerin), NOP56/NOP58, NHP2, GAR1, FTSJ3, and any combination thereof.

4. Factors required for ribosome subunit assembly and maturation, such as NVL/NVL2, NPM1, NOC3L, PDCD11, MDNI/Real, RIOK1, RIOK2, LTVI, TSR1, and any combination thereof.

5. Ribosomal proteins themselves, such as selected structural proteins (e.g., RPS6, RPL5), and other ribosomal proteins that can be directly degraded or downregulated.

6. Export/trafficking factors, such as NMD3, XPOI/CRMI, and other factors required for transport of intermediate ribosomal subunits from the nucleolus to the nucleoplasm and from the nucleus to the cytoplasm.

The above classes and examples of genes, proteins, and molecular factors involved in ribosome biogenesis that can be targeted by a quiescent-inducing agent are for illustrative purposes and are not intended to be limited in scope. In some embodiments, the quiescent-inducing agent has function in targeting at least one ortholog, paralog, or molecule with functional equivalence to any one of the molecules involved with ribosome biogenesis. In some embodiments, the quiescent-inducing agent inhibits function directly (e.g., small molecules, targeted degradation). In some embodiments, the quiescent-inducing agent inhibits function indirectly (e.g., transcriptional silencing, RNA-targeting approaches).

There are many potential applications for an ability to induce cellular quiescence. Such ability would be useful, for example, in fundamental research of cell biology, including studies of stress tolerance and survival, molecular pathways involved in quiescence induction, maintenance, and exit, studies of the capacity of stem cells to self-renew and generate differentiated cell types, or studies of longevity of different cell types. A capacity to induce cellular quiescence would also be useful for biotechnological, clinical, and therapeutic purposes. For instance, the reversible proliferative arrest, onset of hypometabolism, and resistance to stress imparted by quiescence induction could, with or without subsequent freezing or vitrification, improve preservation of cells, tissues, and organs, making these more readily available for transplantations. Similarly, extended preservation of viability and function of such biological materials would make them amenable to manipulation ex vivo for therapeutic purposes, as well as render them better suited to biobanking. Induction of cellular quiescence could also serve the purpose of slowing biological time and aging of whole organisms, inducing their hibernation, stasis, or torpor. Among other, this could render organisms better tolerable to long-lasting travel, improving, for instance, viability of plants and animals during transport, or supporting long-distance space travel of humans.

Organ and tissue transplantation remains constrained by the short window of viability between procurement and implantation. Current preservation methods are insufficient to prevent degradation of high-metabolism tissues such as heart, liver, or lung. Moreover, variability in donor-recipient matching, availability of surgical teams, and logistics across time zones compound the limitations. A method that induces a dormant yet viable state in cells or tissues could mitigate these issues. By slowing metabolic processes and arresting the cell cycle without inducing damage, such a method could substantially lengthen the shelf life of transplantable biomaterials. In turn, this would allow for more precise immunological screening, broader donor sharing across regions, and increased equity in organ access. These needs have been highlighted in recent work underscoring the critical gap between tissue demand and preservation capabilities.

There are few known approaches to induce quiescence of cells of multicellular organisms. Examples include quiescence entry of human fibroblasts upon mitogen withdrawal, contact inhibition, or loss of adhesion. However, depending on the initiating signal, fibroblasts enter different non-proliferative states of uncertain longevity and reversibility, and the same external signals cannot induce quiescence in many other cell types. Given the ubiquity of cellular quiescence as the most prevalent cellular state in nature, its applicative potential, but paucity of options for its manipulation, there is a need for methods to induce quiescence broadly in different cell types.

The detailed description set forth below is intended as a description of various configurations of the subject technology and is not intended to represent the only configurations in which the subject technology may be practiced. The detailed description includes specific details for the purpose of providing an understanding of the subject technology. It will be apparent to those skilled in the art that the subject technology may be practiced without these specific details.

It is to be understood that this invention is not limited to the particular methodology, protocols, cell lines, organism, species or genera, and reagents described, as such may vary. It is also to be understood that the terminology used herein is for the purpose of describing particular embodiments only, and is not intended to limit the scope of the present invention, which will be limited only by the appended claims. Unless specifically defined herein, all terms used herein have the same meaning as they would to one skilled in the art of the present invention. Practitioners are particularly directed to Liu et al, Nat Commun 2025 (PMID: 40533478), Marescal and Cheeseman, Dev Cell 2020 (PMID: 33171109), Cheung and Rando, Nat Rev Mol Cell Biol 2013 (PMID: 23698583), O'Farrell, Philos Trans R Soc Lond B Biol Sci 2011 (PMID: 22084377), Dörner et al, EMBO J 2023 (PMID: 36762427), Hori et al, Nat Rev Mol Cell Biol 2023 (PMID: 36732602), Nabet et al, Nat Chem Biol 2018 (PMID: 29581585), Zhang et al, Comput Struct Biotechnol J 2022 (PMID: 36249565), Morrison et al, Cell 1997 (PMID: 9039255).

### Terms

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The section headings used herein are for organizational purposes only and are not to be construed as limiting the subject matter described.

The articles “a” and “an” are used herein to refer to one or to more than one (for example, at least one) of the grammatical object of the article, unless the context dictates otherwise. By way of example, “an element” means one element or more than one element.

The terms “first,” “second,” and “third” used in combination with substances are intended to designate distinguishable features to similar substances and do not imply any particular order unless otherwise specified.

As used herein, the term “including” as well as other forms such as “includes” and “included” is not limiting.

As used herein, “approximately” and “about” mean that a number or other measure referred to as “approximately” or “about” comprises the recited number plus or minus 1-10% of that recited number. For example, “about” 100 degrees can mean 95-100 degrees or as few as 99-101 degrees depending on the context. Whenever it appears herein, a numerical range such as “1 to 20” refers to each integer in the given range, i.e., meaning only 1, only 2, only 3, etc. up to and including only 20.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it includes, but is not limited to, primates, including simians (chimpanzees, apes, monkeys) and humans, cattle, horses, shecp, goats, swine, rabbits, dogs, cats, rodents, rats, mice, guinea pigs, or the like.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

As used herein, the term “compound” has its usual meaning and thus can refer to a chemical or biological material. Non-limiting examples of a compound include an atom, small molecule, large molecule, peptide, protein, drug, or any combination thereof.

As used herein, the term “composition” has its usual meaning and thus can refer to a chemical or biological compound or substance, or a mixture or combination of two or more such compounds or substances. In some embodiments, the composition comprises an atom, small molecule, large molecule, peptide, protein, salt, buffer, or any combination thereof. In some embodiments, the composition is formulated for a specific use, such as for administration to a subject.

As used herein, the terms “pharmaceutical composition” or “pharmaceutical formulation” refer to a chemical or biological compound or substance, or a mixture or combination of two or more such compounds or substances, intended for use in the cure, treatment, prevention or diagnosis of a disease or symptom.

As used herein, the term “pharmaceutical agent” has its usual meaning and thus can refer to a compound or composition with a known therapeutic function. In some embodiments, that function has use in the cure, treatment, prevention or diagnosis of a disease or symptom. In some embodiments, a pharmaceutical agent comprises a drug. In some embodiments, a pharmaceutical agent comprises a composition, the composition comprising a drug.

As used herein, “carrier” refers to a substance that serves as a vehicle for improving the efficacy of delivery or the effectiveness of a pharmaceutical composition, or both.

As used herein, “pharmaceutically acceptable” when used to define a carrier, whether diluent or excipient, refers to a substance that is compatible with other ingredients in a formulation and does not exert deleterious effects to the recipient thereof. As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also include one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, thritol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

As used herein, the terms “treating” or “treatment” have their plain and ordinary meaning as understood in light of the specification, and refer to an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (e.g., not worsening) the state of disease, prevention of a disease's transmission or spread, delaying or slowing of disease progression, amelioration or palliation of the disease state, diminishment of the recurrence of disease, and remission, whether partial or total and whether detectable or undetectable. Treatment methods include administering to a subject a therapeutically effective amount of an active agent. The administering step may include a single administration or may include a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment of a disease or disorder may increase or decrease over the course of a particular treatment regime. Changes in dosage may result and become apparent by standard diagnostic assays known in the art. In some instances, chronic administration may be required.

“Treat” and “treating” are not limited to the case where the subject (e.g. patient) is cured and the disease is eradicated. Rather, the present disclosure also contemplates treatment that merely reduces symptoms, and/or delays disease progression.

As used herein, the term “therapeutic target” has its plain and ordinary meaning as understood in light of the specification and refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

The term “administering” includes contact with a skin, tissue, mucus, or fluid of a subject. It includes topical contact, administration as a suppository, intravaginal, intravenous, intraperitoneal, intramuscular, intralesional, intra-tumoral, intrathecal, intranasal, or subcutaneous administration, or the implantation of a slow-release device, e.g., a mini-osmotic pump, to a subject. Administration can be by any route unless specifically stated, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intra-tumoral, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein. The preferred administration of the present disclosure comprises contact with a subject's reproductive glands or tissues, such as through transvaginal delivery and/or intravaginal delivery.

As used herein, “excipient” refers to a pharmacologically inactive substance or compound that is formulated in combination with a pharmacologically active ingredient of a pharmaceutical composition, wherein an excipient may include a bulking agent, a filler, a diluent, or a product for drug solubility, drug absorption, or for modulating a pharmacokinetic property of an active ingredient of a pharmaceutical composition.

As used herein, “therapeutically effective amount” refers to the amount or quantity of a pharmaceutical composition that elicits a desired clinical or biological response in a cell, a tissue, an organ, an animal or a human, or an amount or quantity that is clinically useful for reducing, eliminating, or otherwise affecting a disease, condition, or medical complication in a subject.

As used herein, “administration” (or other forms such as “administering”) refers to the act of providing a substance or compound to a subject in need thereof.

The term “active ingredient” as used herein is to be given its ordinary and customary meaning to a person of ordinary skill in the art (and is not to be limited to a special or customized meaning) and refers to a component that confers a function. In some embodiments, the component provides direct or indirect effect in the cure, mitigation, treatment, diagnosis, or prevention of disease or condition in the body of humans or animals.

The term “gene” as used herein, refers to a DNA sequence that comprises control and coding sequences necessary for the production of a polypeptide or protein precursor. The polypeptide can be encoded by a full length coding sequence or by any portion of the coding sequence, as long as the desired protein activity is retained.

“Purified” refers to molecules, including but not limited to nucleic, ribonucleic, lipid or amino acid sequences, which are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. “Cell” means the smallest structural unit of living matter capable of functioning autonomously, consisting of one or more nuclei, cytoplasm, and various organelles, all surrounded by a semipermeable membrane. Cells include all somatic cells obtained or derived from a living or deceased animal body at any stage of development as well as germ cells, including sperm and eggs (animal reproductive body consisting of an ovum or embryo together with nutritive and protective envelopes). Included are both general categories of cells: prokaryotes and eukaryotes. The cells contemplated for use in this disclosure include all types of cells from all organisms in all kingdoms: plants, animals, protists, fungi, archaebacteria and cubacteria. Stem cells are cells capable, by successive divisions, of producing specialized cells on many different levels. For example, hematopoietic stem cells produce both red blood cells and white blood cells. From conception until death, humans contain stem cells, but in adults their power to differentiate is reduced.

“Organ system” refers to a collection of cells, tissues, organs, or any combination thereof that work together in a shared function. Nonlimiting examples of an organ system includes the digestive system, respiratory system, circulatory system, muscular system, skeletal system, endocrine system, exocrine system, lymphatic system, nervous system, and reproductive system. In some embodiments, organ system can refer to a group of cells with a shared function, such as immune cells, endothelial cells, or blood cells.

“Growth media” are compositions used to grow microorganisms or cells in culture. There are different sorts of media for growing different sorts of cells. The biggest difference in growth media are between those used for growing cells in culture (cell culture uses specific cell types derived from plants or animals) and those used for growing microorganisms (usually bacteria or yeast). These differences arise due to the fact that cells derived from whole organisms and grown in culture are often incapable of growth without the provision of certain requirements, such as hormones or growth factors which usually occur in vivo. In the case of animal cells these requirements are often provided by the addition of blood serum to the medium. These media are often red or pink due to the inclusion of pH indicators. Growth media for embryonic stem cells preferably contains minimal essential medium, i.e., Eagle's: amino acids, salts (Ferric nitrate nonahydrate, Potassium chloride, Magnesium sulfate, Sodium chloride, Sodium dihydrogen phosphate), vitamins. (Ascorbic acid, Folic acid, Nicotinamide. Riboflavin, B-12) or Dulbecco's: additionally iron, glucose; non-essential amino acids, sodium pyruvate, beta-mercaptoethanol, L-glutamine, fetal bovine serum and Leukemia Inhibitory Factor (LIF). In the case of microorganisms, there are no such limitations as they are often single cell organisms. One other major difference is that animal cells in culture are often grown on a flat surface to which they attach, and the medium is provided in a liquid form, which covers the cells. Bacteria such as Escherichia coli (E. coli, the most commonly used microbe in laboratories) may be grown on solid media or in liquid media, liquid nutrient medium is commonly called nutrient broth. The preferred growth media for microorganisms are nutrient broth or Luria-Bertani medium (L-B medium). Bacteria grown in liquid cultures often form colloidal suspensions. When agar (a substance which sets into a gel) is added to a liquid medium it can be poured into Petri dishes where it will solidify (these are called agar plates) and provide a solid medium on which microbes may be cultured.

“Prevent” and “preventing” include the prevention of the recurrence, spread or onset. It is not intended that the present disclosure be limited to complete prevention. In some embodiments, the onset is delayed, or the severity of the disease is reduced.

The term “proliferation” as used herein refers to division of cells, either in a population or a subpopulation thereof, and thereby increase in their numbers. Cells may exhibit different rates of proliferation whereby their numbers are increasing slower or faster. Cells may also not be proliferating, for example, if they are quiescent, dead, or undergoing apoptosis. A population of cells is a specific line or type of cells, for example, HEK293T cells or hematopoietic stem cells.

As used herein, the terms “quiescence” and “cellular quiescence” refer to living cells that are not actively proliferating. In some embodiments, quiescence refers to a cell population in which there is at least 10%, 20%, 30%, 40%, 50%, 60%, 70%, 80%, 90%, 95%, 99%, or 100% reduced or arrested proliferation compared to a cell population that is non-quiescent. Cells can be artificially induced to become quiescent or can be naturally quiescent. Quiescent cells are generally regarded as not being in any of the four phases of the mitotic cell cycle, G1, S, G2 and M, and are usually described as being in a G0 state, to indicate that they would not normally progress through the cycle. However, although cells predominantly enter quiescence from G1 phase, they can also enter quiescence (and thus exit cell cycle) from any other phase. Cells can remain quiescent for different periods of time, from a few days to several decades. Cells can generally be induced to enter quiescence by the methods of invention, and cells of specific cell lineages can be induced to quiescence by other methods, including but not limited to, mitogen withdrawal, contact inhibition, or loss of adhesion. Cells can be induced to exit quiescence by removal of the agent used to induce quiescence in the methods of invention, or by other methods, such as contacting the cells with cytokines or growth factors.

As used herein, the term “ribosome biogenesis” is used interchangeably with “ribogenesis” and refers to the process of making ribosomes. Ribosomes have two major components, the small and large ribosomal subunits, with each subunit comprising one or more ribosomal RNA (rRNA) molecules and many ribosomal proteins. Prokaryotes have 70S ribosomes, each comprising a small (30S) and a large (50S) subunit. Eukaryotic cells have 80S ribosomes located in the cytosol, each comprising a small (40S) and large (60S) subunit. The eukaryotic 40S subunit contains 18S RNA and 33 proteins. The eukaryotic large subunit contains 5S RNA, 28S RNA, 5.8S RNA and 49 proteins. Ribosome biogenesis involves several key steps: (a) transcription of rRNA, which in eukaryotic cells occurs in the nucleolus and is performed by RNA polymerase I (Pol I) for the 47S precursor rRNA (pre-rRNA) transcript from which 18S, 5.8S, and 28S rRNAs are produced; the 5S rRNA is transcribed separately by RNA polymerase III; (b) processing of rRNA, which is extensive and includes numerous cleavages, chemical modifications, and folding to form mature rRNAs; cleavages of the 47S pre-rRNA are carried out by numerous endoribonucleases (including RNase MRP, LASIL, UTP23, UTP24, NOB1, RCL1, and others) and exoribonucleases with help of additional proteins; (c) synthesis of ribosomal proteins, which are produced in the cytoplasm and then imported into the nucleolus, where they assemble with rRNAs; (d) assembly of ribosomal subunits, which occurs in a highly orchestrated manner to form the 40S and 60S subunits in eukaryotic cells and 30S and 50S subunits in prokaryotes; (e) export of the assembled ribosomal subunits from the nucleolus to the cytoplasm (in eukaryotes) or from the nucleoid to the cytoplasm (in prokaryotes); (f) final assembly and functional activation, which occurs in the cytoplasm by the small and large ribosomal subunits coming together during translation initiation to form functional ribosomes capable of synthesizing proteins. Ribosome biogenesis is a highly regulated and energy-consuming process, involving participation of more than 400 proteins, and is critical for cell growth and proliferation.

The methods of the present disclosure are based on inhibition of ribosome biogenesis and are useful in any situation in which induction of cellular quiescence is desirable. The methods are also useful in any situations where it is desirable to increase cellular stress tolerance and survival, or reduce cellular metabolism (including but not limited to reduction in the rate of cellular ATP production, gene transcription, protein biosynthesis, DNA replication, signaling and other metabolic pathways) without necessarily inducing cellular quiescence. The methods of the disclosure may be used for such purposes where, by way of non-limiting examples, cells are a priori not dividing, as is the case for post-mitotic cells, including but not limited to differentiated or senescent cells, or cells that are only temporarily not dividing, including quiescent cells. In the latter case, the methods of the disclosure are useful for maintenance rather than induction of cellular quiescence, including, for example, quiescence of stem cells, such as skeletal muscle stem cells, hematopoietic stem cells, or neural stem cells. The methods of the disclosure are further useful for situations where it is desirable to slow down that rate of cell division with no induction of cellular quiescence (the latter implying a complete temporary arrest of cell division), situations where arrest of cell division and maintained cell survival is desirable but with no need for reversibility of the proliferative arrest, or to induce death of certain cancer cells that cannot sustain or be induced into quiescence upon inhibition of ribogenesis.

By way of non-limiting examples, the methods of the invention can be used for a variety of purposes in biomedical research as well as for biotechnological, clinical, therapeutic, or exploratory purposes. The methods described herein can be used but are not limited to basic or applied research of cell differentiation, tissue, organoid, organ, and organismal development, aging and longevity, stress tolerance and survival, tissue and organ regeneration, wound healing, fertility, immune responses, and other biological processes where cellular quiescence plays a critical role. The methods of the invention are further useful in situations where improving preservation (including viability and maintaining function) of cells, tissues, organoids, and organs, either per se or in combination with lowering temperature, including but not limited to lowering temperature below the normal range (typically below 35° C. or 95° F.), freezing, or vitrification, is desirable. Non-limiting examples where improving preservation of the said biological materials using the methods of the invention can be useful include transplantations (referring the act or process of transferring cells, tissues, organoids, or organs from one part of a body to another, or from one individual to another, or of in vitro-grown three-dimensional structures into an organism with a typical purpose to replace or repair damaged or malfunctioning organs, tissues, or cells in the recipient) with or without prior ex vivo genetic manipulation, biobanking (referring to the process of collecting, storing, and managing biological materials along with associated health data and information for use in medical research and clinical studies), preservation of stem cell potency by maintaining their quiescent state ex vivo, preservation of reproductive tissues, germ cells, and other cell lineages of plants and animals for breeding purposes, transport between institutions or industries, or for conservation of endangered species.

As used herein, the term “degradation tag (dTAG) system” refers to a chemical biology system for target-specific protein degradation. The dTAG system enables an on-demand immediate and selective degradation of a protein of interest. Here, a degradation tag, KFBP12F36V, is expressed, either ectopically or endogenously from a locus in the genome, in-frame with a protein of interest. A cell-permeable heterobifunctional degrader molecule, termed herein “dTAG” (for instance, dTAG-13 or dTAGY-1), designed to pair the degradation tag with the ubiquitin-proteasome system, can then be used for on-demand, immediate and selective degradation of the protein of interest.

By way of non-limiting example, the dTAG system presents a chemical biology-based approach for targeted degradation of proteins that participate in ribosome biogenesis (either via inhibition of rRNA biogenesis, biosynthesis of ribosomal proteins, or other means) in order to induce their quiescence. Other approaches for targeted protein degradation that can be used to inhibit ribosome biogenesis for the purpose of inducing cellular quiescence include but are not limited to any techniques that harness the power of natural protein degradation systems, such as the ubiquitin-proteasome system or the lysosomal system, for direct degradation of the protein of interest. Without limitation, such approaches include re-engineering of E3 ligases to re-orient their targeting specificity for the protein of interest, fusions of proteins of interest with degrons, that is, specific amino acid sequences that can be recognized by intracellular proteases (degrons including constitutive degrons, phospho-degrons, small molecule-induced degrons, SMASh tags, temperature-induced degrons, light-induced degrons), fusions of proteins of interest with tags to allow for protein degradation by tag-specific degraders (examples of such tags include but are not limited to HaloTag, IKZF3, and the aforementioned KFBP12F36V), use of chimeric molecules linking E3 enzymes to the proteins of interest (including but not limited to PROTACs (Proteolysis Targeting Chimeras)), molecular glues (i.e., small molecules that induce or stabilize interactions between E3 ligases and targeted proteins), Trim-Away approaches (based on introduction into cells of protein-specific antibodies or their derivatives and recognition of the Fc region of the antibody by the cellular E3 ligase TRIM21), and approaches for targeted protein degradation based on the lysosomal pathway (including technologies such as LYTAC, chaperone-mediated autophagy-based, AUTAC-based, ATTEC-based, and AUTOTAC-based protein degradation systems). The present disclosure does not claim composition-of-matter rights over the cited degrader molecules or targeting constructs, but rather their potential use in the disclosed methods.

In certain embodiments, induction of cellular quiescence can be accomplished by genetic approaches to silence expression of genes involved in ribosome biogenesis. Genetic approaches involve methods that aim to reduce or eliminate the expression of a specific gene or genes in a targeted manner. These approaches include but are not limited to, for example, RNA interference (RNAi), which is a sequence-specific post-transcriptional gene silencing mechanism that is normally triggered by double-stranded RNA or endogenous microRNA precursors (pre-miRNAs). This method can be used to knock down gene expression transiently, for example, by introduction to cells of 21-23-nucleotide small interfering RNAs (siRNAs), or stably, for example, by expression of synthetic short hairpin RNAs (shRNAs). Expression of shRNA can also be rendered inducible, for example, by use of inducible promoters that are responsive to an inducer molecule, such as IPTG or tetracycline or its derivatives. Another genetic approach to silence expression of genes participating in ribosome biogenesis involves use of a CRISPR-Cas system to reduce or eliminate expression of the targeted gene by acting either on the DNA or RNA level. Non-limiting examples include use of CRISPR-Cas9 to edit genomic DNA, use of CRISPR interference (CRISPRi) via catalytically dead version of the Cas protein (for example, dCas9 or dCas12a) to impede gene transcription by the RNA polymerase, and use of CRISPR-Cas13 to silence gene expression by targeting and manipulating RNA. Silencing gene expression using any CRISPR-Cas system can also be made transient, stable, or inducible. A further example of the said genetic approaches is use of antisense oligonucleotides (ASOs), which are nucleic acid molecules designed to bind complementary RNAs, including mRNAs and non-coding RNAs, to prevent translation or induce degradation of the targeted RNA, thereby reducing gene expression. Further examples include of a gene involved in ribosome biogenesis, such as DNA methylation or histone modification, to silence its expression. These modifications do not alter the DNA sequence but can regulate gene activity.

In certain embodiments, induction of cellular quiescence can be accomplished pharmacologically by treatment of cells, tissues, organoids, organs, or organisms with a chemical agent that inhibits proteins that participate in ribosome biogenesis. Examples include inhibition of rRNA transcription by RNA polymerase I using known compounds such as cisplatin, oxaliplatin, 5-fluorouracil, methotrexate, actinomycin D, doxorubicin, mitoxantrone, mitomycin C, campthotecin, irinotecan, etoposide, ellipticines, CX-3543, CX-5461, and BMH-21. Another example includes inhibition of rRNA-processing enzymes, such as LASIL, using compounds like HEN-463. The present disclosure does not claim new chemical entities or composition-of-matter claims beyond these named examples. Future development of novel compounds targeting ribosome biogenesis proteins may be subject to separate intellectual property filings.

In some embodiments, induction of cellular quiescence can be achieved by introducing into cells peptides that inhibit the function of cellular proteins or multiprotein complexes required for ribosome biogenesis. As a non-limiting example, this includes inhibition of pre-rRNA transcription by RNA polymerase I using reported peptides derived from the human POLRIF (RPA43) and POLRIG (PAF49) proteins, respectively, and fused to a protein transduction domain such as HIV-1 TAT for intracellular delivery. Novel peptide derivatives, truncations, modifications, or delivery-enhanced constructs based on these sequences are not claimed herein and may be the subject of future development.

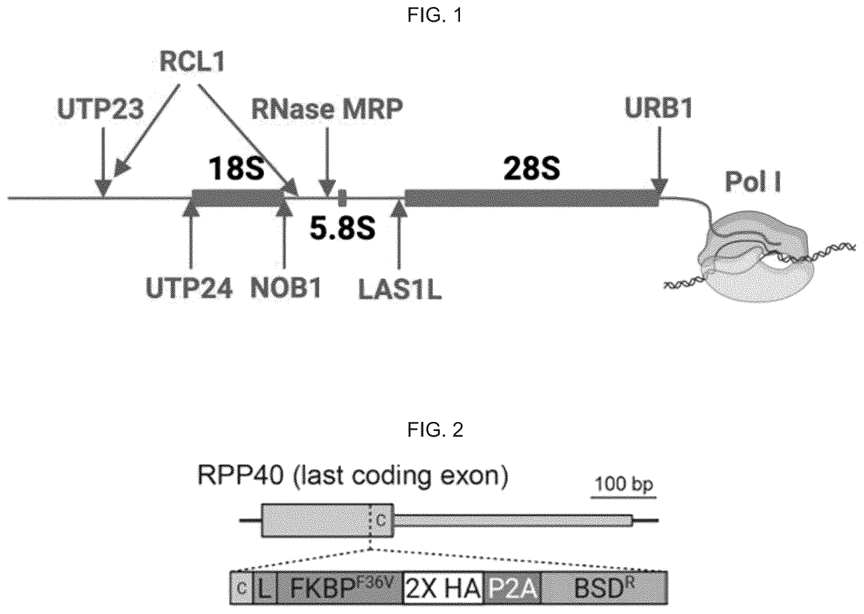

In certain embodiments, induction of cellular quiescence can be accomplished genetically by blocking the sites on pre-rRNA targeted for cleavage by endonucleases, such as RNase MRP, LASIL, UTP23, UTP24, NOB1, RCL1, and others, that participate in normal pre-rRNA processing to form mature rRNAs (FIG. 1), thus inhibiting the cleavage. By way of a non-limiting example, blocking of a specific endonuclease-targeted site on pre-rRNA for the purpose of inducing cellular quiescence can be accomplished by introduction to cells of ASOs that target a region on pre-rRNA that is directly targeted by an endonuclease, and/or a vicinal or distal region on pre-rRNA that is otherwise required for cleavage by the endonuclease. The ASOs can carry various modifications to enhance the binding of ASOs to cognate sequences in pre-rRNA, increase resistance to degradation by nucleases, improve pharmacokinetic characteristics, reduce pro-inflammatory effects, optimize ASO delivery, or for other purposes (Crooke, et al., Nat Rev Drug Discov 20:427-453, 2021; Crooke, et al., J Biol Chem 296:100416, 2021). Such modifications can include but are not limited to modifications of the phosphodiester backbone, including phosphorothioate (PS) linkages and modifications in phosphorodiamidate morpholino oligomers (PMOs), and modifications of the 2′ position in the sugar, including the 2′-O-methoxyethyl (2′-MOE) group and modifications in which the sugar ring is constrained by tethering the 2′ substituent to the 4′ position of the ring, yielding locked nucleic acids (LNAs) or ASOs containing a 2′-constrained ethyl (2′-cEt) moiety.

In some embodiments, the methods of the invention comprise the steps of: (a) administration to cells, tissues, organoids, organs, or organisms of a quiescence-inducing genetic agent, chemical compound, peptide or protein in the appropriate form and concentration for inhibition of ribosome biogenesis; by way of non-limiting examples, this can entail direct addition of the quiescence-inducing agent to cells grown in culture in their full growth media or to the media or another solution used for incubation, preservation, or perfusion of a tissue, organoid, or organ; (b) incubation of the said biological materials with the quiescence-inducing agent until the onset of quiescence or other desired effect, including but not limited to reduction in the rates of cellular metabolism or cell proliferation, or attainment of increased stress tolerance, which may, without limitation, occur within the first four days of incubation, for example, at least about 12 hours, at least about 24 hours, at least about 36 hours, at least about 48 hours, at least about 72 hours, at least about 96 hours; (c) maintenance of quiescence or other attained cellular state by continuous incubation of the said biological materials in the presence of the quiescence-inducing agent, and its replenishment, as required, for a desired period of time; by way of non-limiting example, the induced cellular quiescence can be maintained for various periods of time from 2 days to 14 days, or longer (e.g., from 2 days to 5 days, or from 3 days to 7 days, or from 3 days to 14 days, or longer than 14 days); (d) release of the said biological materials from the induced cellular quiescence to allow cells to re-enter proliferation, which can, in some embodiments, be achieved by removal of the quiescence-inducing agent through washing with media, physiologic or other solutions, omitting replenishment of the quiescence-inducing agent, or other appropriate means; release from other attained cellular states, except cell death, can be achieved in a similar manner. In some embodiments, recovery from quiescence may be facilitated or promoted by providing the cells, tissues, organoids, tumors, organ systems, or organisms with a nutrient-enriched medium, for example by increasing concentrations of amino acids, glucose, growth factors, or other supplements known to enhance cellular metabolism and proliferation. Inhibition of ribosome biogenesis and the attainment of the desired cellular state, which is preferably but not necessarily quiescence, can be assessed by a variety of standard approaches, including but not limited to the analyses of rRNA biogenesis by northern blotting, fluorescence in situ hybridization (FISH), quantitative polymerase chain reaction (qPCR), analyses of the cell cycle, viability, DNA synthesis (for instance, via incorporation of EdU), gene transcription (for instance, via incorporation of EU), and mitochondrial activity (for instance, via staining by MitoTracker dyes) by flow cytometry, changes in cell numbers by cell counting and evaluation of cell death by Trypan blue staining, analyses of translational rate by the puromycin incorporation assay, measurements of the rate of ATP production by the ATP rate assay using Seahorse analyzer (Agilent).

### EXAMPLES

The following examples merely illustrate some of the preferred modes now contemplated for practicing the invention, but should not be construed to limit the invention. It is also to be understood by those of skill in the art that numerous alterations and changes can be made in the particular embodiments exemplified without departing from the intended scope of the invention. Such alterations and changes are intended to be included within the scope of the appended claims.

### Example 1

This example describes the use of the degradation tag (dTAG) chemical biology system in different human cells for targeted degradation of a subunit of RNase MRP, a conserved enzyme critical for rRNA biosynthesis, leading to induction of cellular quiescence. Nonlimiting examples of methods to characterize compromised rRNA biogenesis and induction of cellular quiescence are also described. These results were previously described in Liu et al., Nat Commun 2025 (PMID: 40533478), which is incorporated herein in its entirety.

Biogenesis of rRNAs requires nucleolytic cleavage by several enzymes, among them RNase MRP, which is conserved among eukaryotes, that splits pre-rRNA into segments destined for incorporation into the small or large ribosomal subunits (FIG. 1). RNase MRP comprises a unique catalytic RNA (ribozyme) and several protein subunits. Previous attempts at inhibiting RNase MRP activity have met with problems associated with inefficient depletion or low cell numbers. Given its essential cellular role, human RNase MRP was selected for rapid depletion by directly targeting its protein subunits using the conditional dTAG approach (FIG. 2). Tagging just one subunit was found to be sufficient for enzymatic inhibition, since depletion of virtually any of the RNase MRP protein subunits reduces the levels of the associated catalytic RNA in cells.

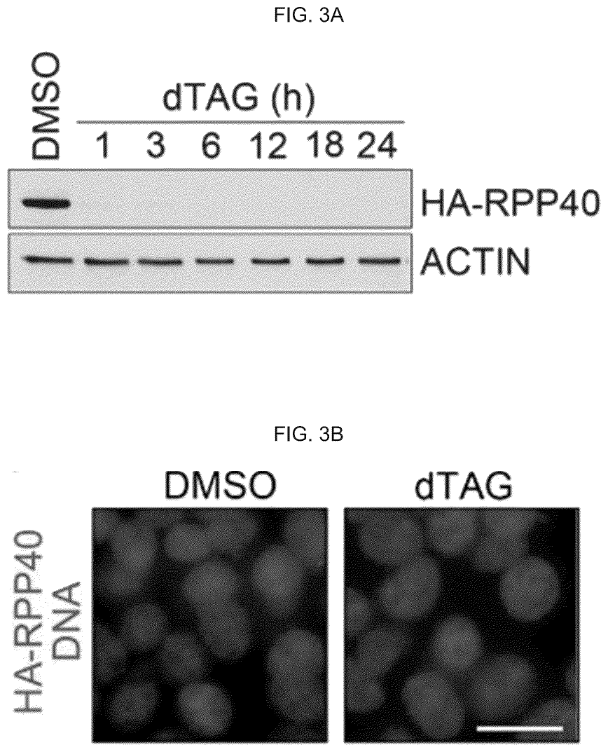

Using homozygous knock-in of FKBP12^{F36V}, HEK293T cells were engineered and maintained in DMEM supplemented with 10% FBS (Cytiva, SH30910.03) and 100 U/ml Penicillin-Streptomycin (Gibco, 15140122) at 37° C. and 5% CO₂, for targeted degradation of the protein subunit of RNase MRP, RPP40 (FIG. 2). To do this, a FKBP^{F36V}-containing knock-in cassette was introduced homozygously into the last coding exon of the RPP40 gene. Modified plasmids pCRIS-PITChv2-dTAG-BSD (Addgene, 91795) and pX333 (for simultaneous delivery of the PITCh and gene-targeting gRNAs; Addgene, 64073) were introduced into the cells via transfection to generate the homozygous ‘C40’ cell line. Treatment of C40 cells with a combination of cell-permeable degraders, dTAG-13 at 500 nM final concentration and dTAG^{V-}1 at 1000 nM final concentration (henceforth dTAG), led to potent, rapid, and sustainable degradation of the targeted subunits, as determined by western blotting and immunofluorescence analyses (FIGS. 3A-3B). For the western blotting (FIG. 3A), whole cell lysates were run on a 15% SDS-polyacrylamide gel and transferred to supported nitrocellulose membrane (Bio-Rad) at 145 V for 1.5 h. The membrane was then blocked for 1 hour in 5% non-fat dry milk in TBST (137 mM NaCl, 20 mM Tris, pH 7.6, 0.1% Tween-20), rinsed three times for 5 min with TBST, and incubated with primary antibody (1:1000 in 3% BSA in TBST) overnight at 4° C. The blot was washed in TBST 5 min for 3 times, incubated with HRP-conjugated secondary antibodies in 5% milk in TBST for 1 hour and then washed again before developing. Beta-Actin was detected by incubating the blot with HRP-conjugated beta-actin antibody (1:15,000 in 5% milk in TBST) for 15 min followed by washing and developing. HRP signal was detected by developing with SuperSignal™ West Pico PLUS Chemiluminescent Substrate (Thermo Scientific, 34578). For immunofluorescence (FIG. 3B), cells were fixed in 4% formaldehyde in PBS for 10 min at room temperature, permeabilized in 0.5% Triton X-100 in PBS, blocked in 5% FBS in PBS, and probed with primary antibodies at 4° C. for overnight. After an overnight incubation, the cells were probed with fluorochrome-conjugated secondary antibodies for 1 hour at room temperature and mounted using VECTASHIELD Antifade Mounting Medium with DAPI (Vector Laboratories, H-1200-10). Images were taken with the LEICA DMi8 and LSM 880 confocal microscope (Zeiss).

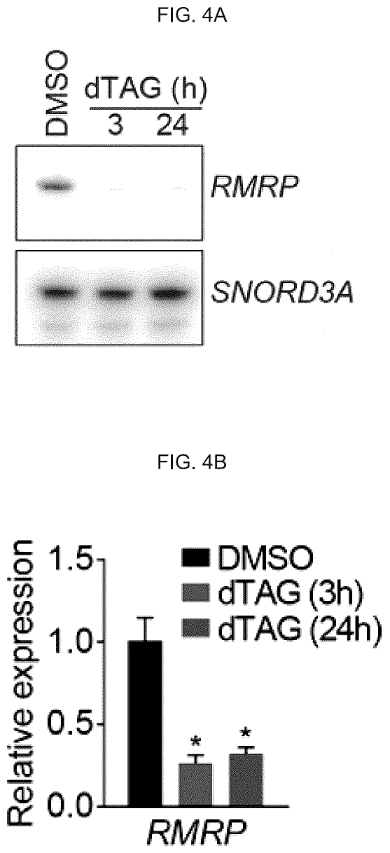

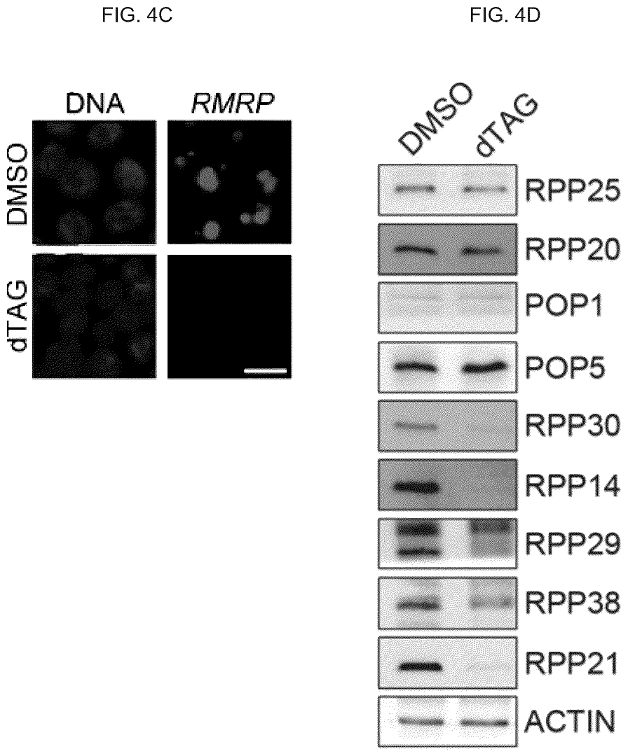

Notably, dTAG treatment of C40 cells caused a rapid decrease in the levels of the catalytic RNA of RNase MRP (RMRP), as determined by northern blotting, qPCR, and fluorescence in situ hybridization assays, consistent with the essential role of protein subunits stabilizing the catalytic RNA (FIGS. 4A-4C). The probe for the northern analysis (FIG. 4A), was a DNA oligonucleotide (ThermoFisher) 5′ end-labeled with 32P gamma-ATP (PerkinElmer, NEG502A250UC) and T4 polynucleotide kinase (New England Biolabs, M0201L) according to the manufacturer's instructions. About 5 μg of total RNA was separated on a 10% polyacrylamide/7 M urea/1×TBE gel. RNA was transferred to a Hybond N+ membrane (Cytiva, RPN203B) in 1×TBE at 100 mA for 1 h at cold room 4° C. and then cross-linked twice at 1200 mJ/cm² before prehybridization in 10 mL of hybridization buffer (Invitrogen, AM8670) for 60 min at 42° C. The probe was hybridized with the membrane overnight at 42° C. The next day, the membrane was washed sequentially with the wash buffer 1 (2×SSC, 0.1% SDS), wash buffer 2 (1×SSC, 0.1% SDS), and wash buffer 3 (0.1×SSC), once each for 30 min. The membrane was then developed using the storage phosphor screen (GE Healthcare, 0146-931) in an autoradiography cassette (Fisher Scientific, FBAC810) overnight at −20° C. The next day, the phosphor screen was imaged with Typhoon imager (Cytiva). For the qPCR analysis (FIG. 4B), total RNA was extracted from samples using TRIzol Reagent (ThemoFisher, 15596018) and Direct-zol RNA Miniprep (Zymo Research, R2050) according to the manufacturer's instructions. cDNA was prepared from equal amounts of RNA using PrimeScript RT Reagent Kit (Takara, RR037A) following manufacturer's instructions. The qPCR was performed using PowerUp SYBR Green Master Mix (ThermoFisher, A25742) and oligonucleotide primers to amplify the cDNA on the CFX Connect Real-Time PCR Detection System at the annealing temperature of 63° C. Relative mRNA levels were normalized to relative expression levels of the SNORD3A gene that was used as an internal control. For the RNA fluorescence in situ hybridization assay (FIG. 4C), a RMRP-cy5 FISH probe was ordered from Integrated DNA Technologies.

A further inspection of the enzymes' components by western blotting revealed that loss of RPP40 led to co-depletion of several other subunits of RNase MRP, conceivably reflecting a stepwise assembly of RNase MRP (FIG. 4D).

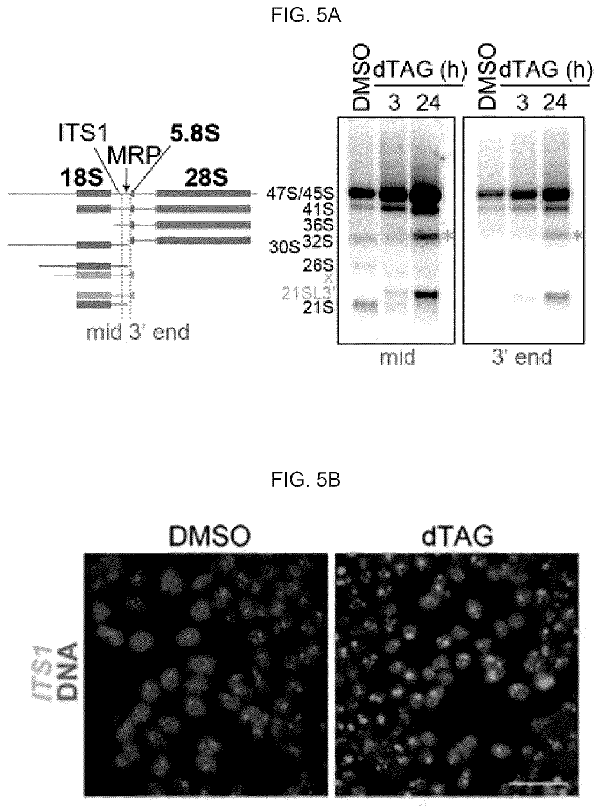

Next, it was asked how the abruptly reduced level of the catalytic RNA in dTAG-treated C40 cells affected rRNA processing. Northern blot analyses revealed a substantial, time-dependent accumulation of 47S/45S rRNA and other RNA precursors consistent with a major defect in cleavage of the ITS1 segment by RNase MRP, blocking downstream rRNA processing of 18S, 5.8S, and 28S rRNA (FIGS. 5A-5B). Together, these results establish a system for rapid, inducible ablation of rRNA processing in human cells.

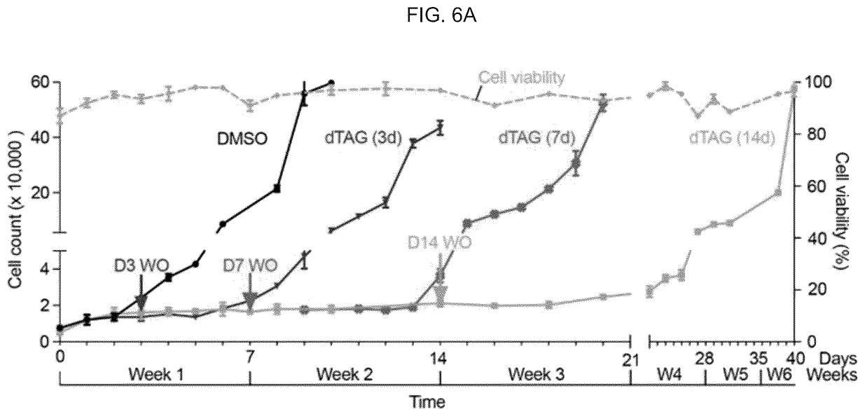

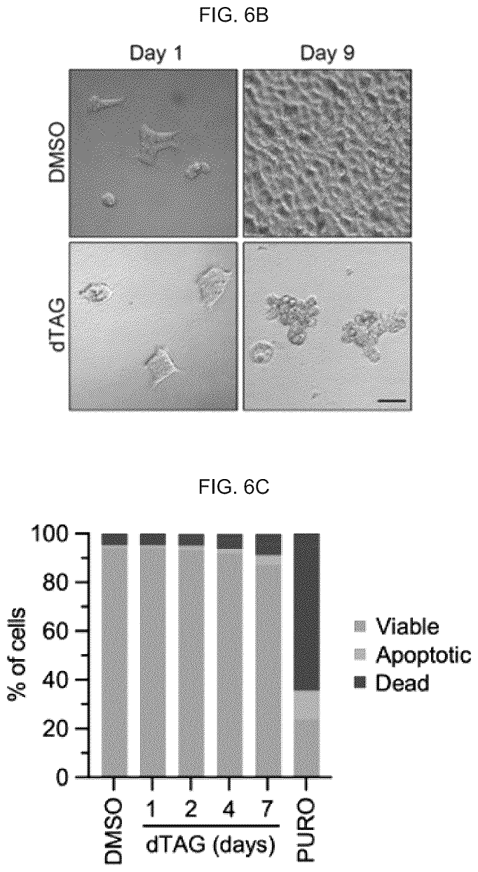

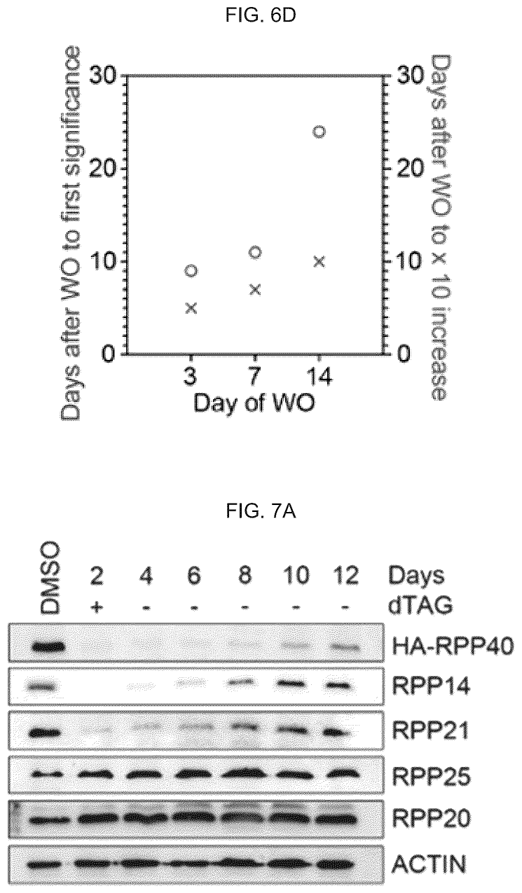

Tight control of rRNA biogenesis is critical for maintenance of cellular homeostasis and survival. It was therefore considered how these parameters may be affected by an abrupt depletion of RNase MRP in human cells. Surprisingly, it was found that depletion of RNase MRP maintained cell survival but caused a complete proliferative arrest between day 2 and 3 (FIG. 6A). To monitor the rate of cell growth, cells were seeded at 5,000 cells per well in a 48-well plate. At each indicated time point, cells were harvested by digestion with 0.25% trypsin-EDTA (Gibco, 2661762) from individual wells, and then counted using a hemocytometer (Fisher Scientific, 0267151B). Cell viability was determined via staining with Trypan Blue (Sigma, T8154-100ML). To wash out dTAG from cultures, cell culture supernatant was removed, attached cells were harvested by digestion with 0.25% trypsin-EDTA, combined with the supernatant, and washed twice with complete media. Cells were then seeded into a new 48-well plate and counted at each indicated time point.

The arrested C40 cells showed rounded morphology and weaker surface attachment compared to their vehicle-treated controls, but remained viable for several weeks (FIGS. 6A-6C). Strikingly, removal of dTAG after different times of treatment allowed the arrested cells to resume proliferation, with the delay before the proliferative re-start correlating with the time of dTAG removal (FIGS. 6A and 6D). The observed proliferative arrest and its reversibility are consistent with the defining properties of cellular quiescence.

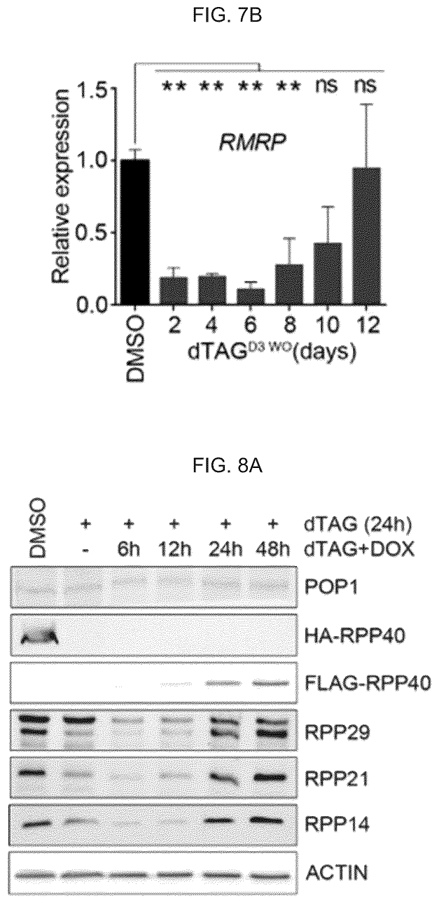

It was next asked how levels of the multiple RNase MRP subunits might correlate with timing of the proliferative arrest and its reversal. Notably, unlike the rapid, dTAG-induced depletion of the enzymatic components (FIGS. 3A-3B and 4A-4D), restoration of their levels upon dTAG removal required a considerably longer time (FIGS. 7A-7B). Interestingly, whereas cell division did not cease until several days of depletion of RNase MRP, resumed proliferation was observed roughly coincident with re-appearance of the bulk of each enzyme (FIGS. 6A and 7A-7B).

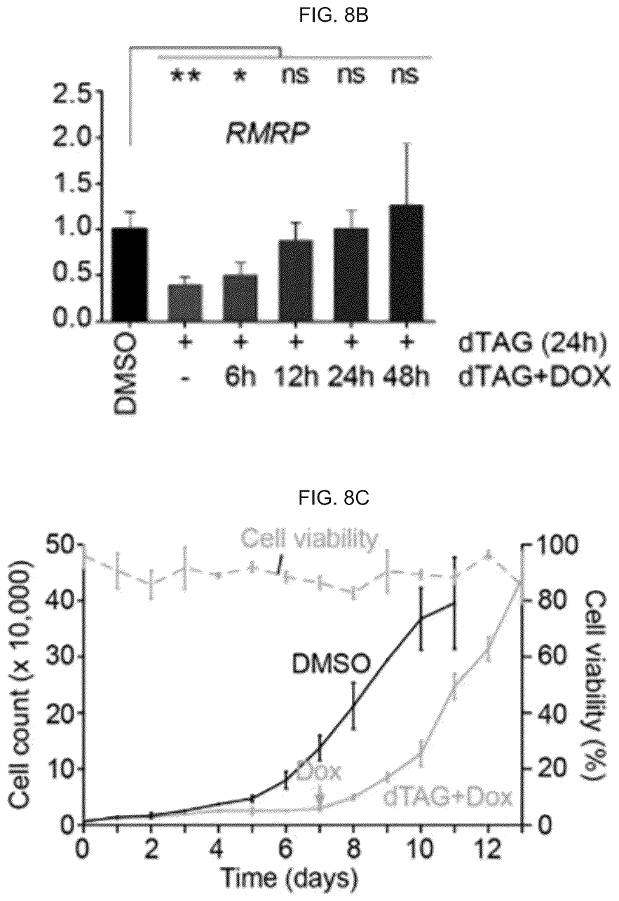

To validate that the restored levels of enzymatic components and resumed cellular proliferation indeed relied on expression of the genetically targeted RPP40, a C40 cell line was derived with doxycycline (Dox)-inducible expression of ectopic, Flag-tagged RPP40 via lentiviral infection of C40 cells, using the pLIX-403 plasmid (Addgene, 41395; FIGS. 8A-8C). Despite a somewhat weaker depletion of RNase MRP in these cells, presumably due to slight leakiness of the Dox-inducible cassette, dTAG treatment efficiently arrested their proliferation (FIGS. 8A-8C). Importantly, addition of Dox during continued presence of dTAG promptly restored the levels of the enzymatic components as well as cellular proliferation (FIGS. 8A-8C). It was concluded that depletion of RNase MRP can cause human cells to enter quiescence.

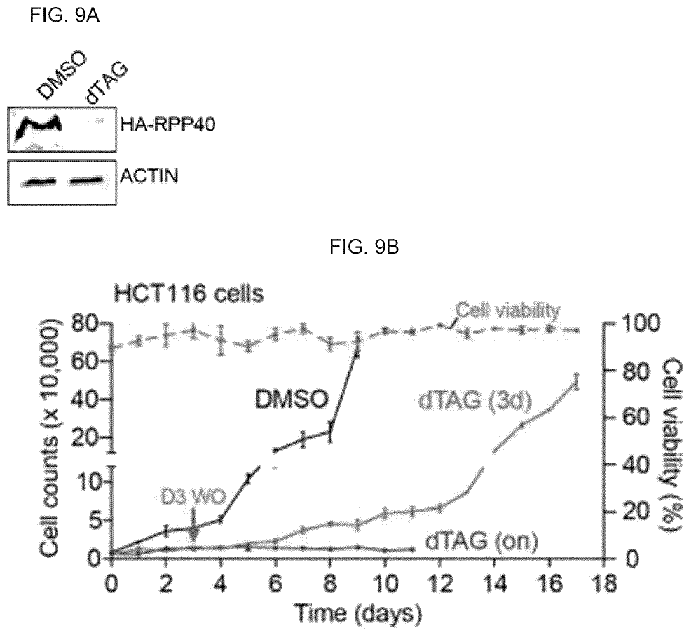

It was also determined whether other cultured cells could be induced into quiescence in a similar manner. The colorectal carcinoma cells HCT116 were engineered for dTAG-dependent degradation of RPP40, as above (FIGS. 2 and 9A). Analogously to C40 cells, the modified HCT116 cells underwent a complete proliferative arrest upon dTAG treatment, remained viable, and resumed proliferation, though at a later time after washout compared to C40 cells (FIG. 9B). Together, these results suggest that depletion of RNase MRP can induce quiescence in human cells of different lineages.

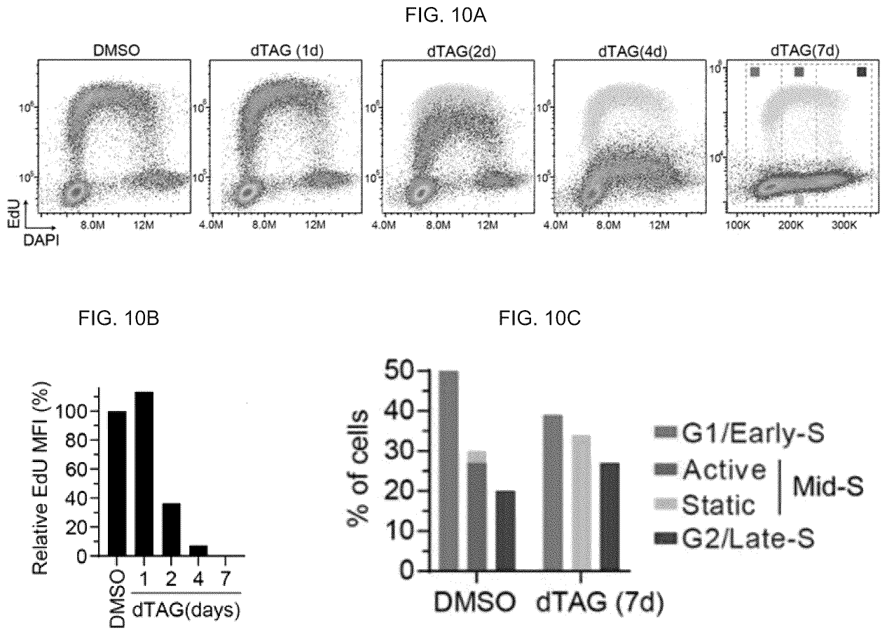

Cells of various organisms predominantly enter quiescence from G1 phase of the cell cycle, although in certain settings quiescence can also be initiated from other cell cycle phases. An EdU incorporation assay in dTAG-treated C40 cells revealed a synchronous and time-dependent reduction in the rate of DNA synthesis, starting at 2 days of treatment; DNA synthesis rate was reduced to <10% at 4 days and completely ceased by 7 days of treatment (FIGS. 6A and 10A-10B). In this assay, samples were analyzed via flow cytometry by resuspending cells in FACS Buffer (5% FBS, 1 mM EDTA pH 8.0 in DPBS). Data was acquired with NovoExpress (v1.5) on NovoCyte 2100Y/Quanteon cytometers and analyzed with FlowJo (v9, v10). Cells were gated using forward and side scatter gating to exclude dead cells and debris, followed by height and area gating to exclude doublets. Cell cycle analysis was performed using the Click-iT Plus EdU kit (ThermoFisher, C10646). At indicated times, dTAG-treated and control cells were pulse-labelled with 10 μM EdU for 1 h. One million cells were collected and fixed with 4% formaldehyde and permeabilized with a saponin-based wash. EdU was labeled in a reaction cocktail with Alexa Fluor 594 or 647 picolyl azide for 30 min at room temperature. After labeling, total DNA content was stained with FxCycle Violet Stain (Invitrogen, F10347) in FACS Buffer for 30 minutes at room temperature and analyzed by flow cytometry. Cell cycle phases were determined based on DNA content and EdU signal. Relative mean fluorescence intensities (MFI) were calculated by subtracting the mean EdU intensity of unstained cells from the mean EdU intensity of stained cells in Mid-S phase at each time of dTAG treatment. Strikingly, even at 7 days, a large fraction of cells, about 35%, contained a DNA amount typical of S-phase cells with a broad distribution between 2C and 4C DNA content (FIGS. 10A and 10C). Thus, driven by the depletion of RNase MRP, HEK293T cells enter quiescence from multiple points in the cell cycle, with a substantial number of S-phase cells becoming quiescent. It was hypothesized that the relative increase in the DNA content of quiescent C40 cells might be due to absence of G1 arrest coupled with reduced ability of cells in S phase to complete genome replication and divide.

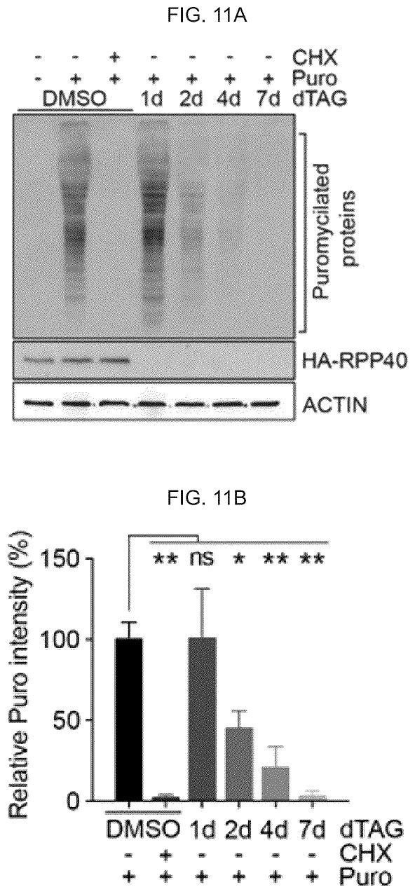

Changes in other critical cellular functions were monitored as the HEK293T cells became quiescent. Quantification of global translation via labeling of nascent polypeptides revealed a sustained rate in the first 24 h of depletion of RNase MRP, after which translation declined precipitously to less than 5% of the initial rate by day 7 (FIGS. 11A-11B). To detect nascent polypeptides, C40 cells at 50% confluency were treated with 1 μg/mL puromycin for 30 min. As a negative control, CHX treatment was done at 10 μg/mL 10 min prior to adding puromycin. Cells were then harvested for SDS-PAGE and western blot analyses and blotted with the anti-puromycin antibody. Samples were tested in biological triplicates and image acquisition was performed with a ChemiDOC imaging systems (Bio-Rad). ImageJ software was used to quantify the intensity of signals. Negative control lane's intensity was considered as background and was subtracted from other lanes' intensity. The subtracted values were normalized to ACTIN and used for relative quantification. The observed changes suggest that limited resources rather than rapid signaling may downregulate translation upon abrupt blockage of rRNA processing.

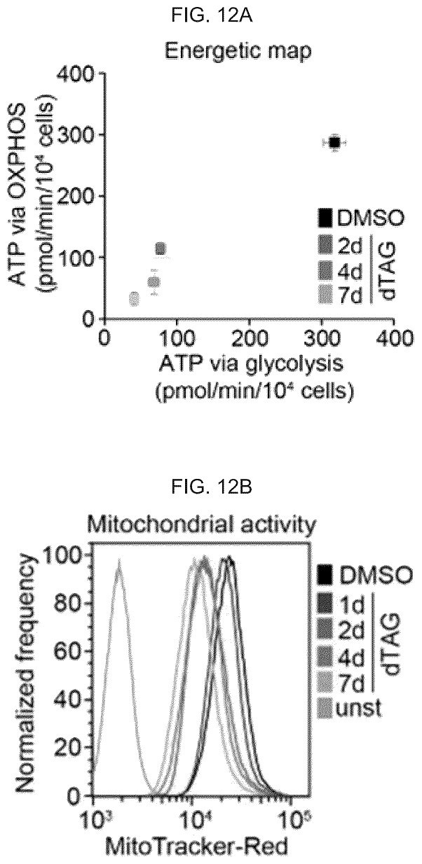

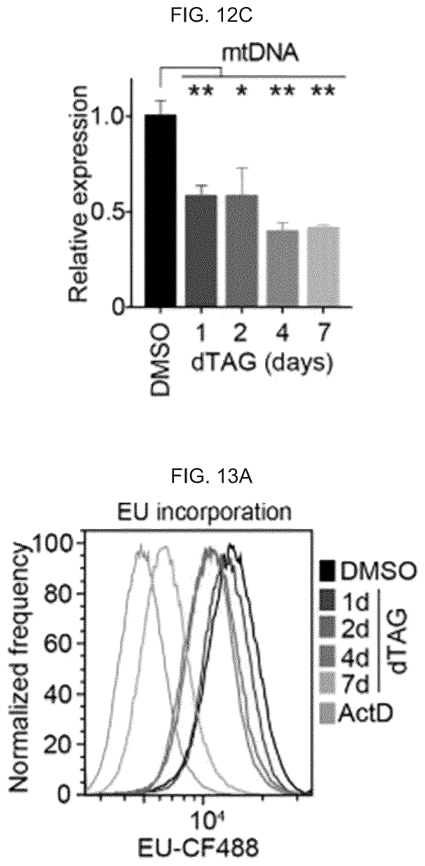

Proliferating cells generally exert a higher metabolic rate compared to their quiescent counterparts. To assess how depletion of RNases P and MRP might affect cellular metabolic activity, the rate of ATP production was measured by dTAG-treated C40 cells. Compared to the decline in translation, milder reduction in total ATP production was observed, which in quiescent cells after 7 days of treatment was reduced to 12% of the initial rate (FIG. 12A). The rates of ATP production via oxidative phosphorylation and glycolysis were comparably reduced, suggesting the cells maintained connectivity between the two main ATP-generating pathways (FIG. 12A). These analyses of ATP production were performed using Seahorse technology, where extracellular acidification rate (ECAR) and oxygen consumption rate (OCR) were measured on a Seahorse XF Pro analyzer (Agilent) following the manufacturer's instructions. In brief, DMSO- or dTAG-treated C40 cells were seeded at 2.5×10+ cells per well in Seahorse XFe96/XF Pro PDL plates. The next day, cells were equilibrated for 1 h in XF assay medium supplemented with 1 mM pyruvate, 2 mM glutamine, and 5 mM glucose in a non-CO₂ incubator. OCR and ECAR were monitored at baseline and after the injections of Seahorse ATP Real-Time Rate Assay Kit reagents: Oligomycin (final concentration 1.5 μM) and Rotenone/Antimycin (final concentration 0.5 μM). Data were analyzed with Seahorse Wave Pro (10.1.0.1). Furthermore, MitoTracker staining and measurement of mtDNA content showed evidence of reduced mitochondrial activity in the arresting cells, consistent with the reduced oxidative phosphorylation (FIGS. 12B-12C). For this assay, mitochondria were labelled using the MitoTracker Red CMXRos (Invitrogen, M7512); after the indicated times, dTAG-treated and control cells were pulse-labelled with MitoTracker for 30 min. One million cells were collected and fixed with 4% formaldehyde and permeabilized with a saponin-based wash. After permeabilization, DNA was stained with FxCycle Violet Stain (Invitrogen, F10347) in FACS Buffer for 30 minutes at room temperature before analysis by flow cytometry. Unstained cells were used as a negative control.

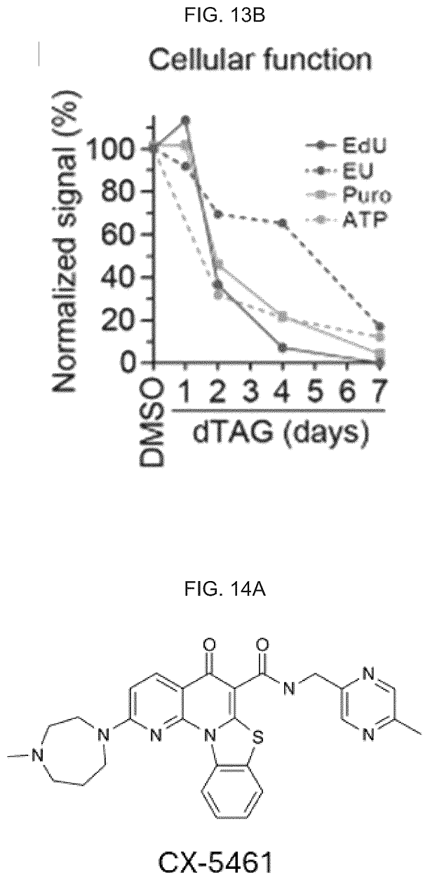

Quiescent cells must retain a basal transcriptional capacity to support important cellular functions necessary for survival. Overall transcriptional activity of dTAG-treated C40 cells were evaluated by labeling newly transcribed RNA with 5-ethynyluridine (EU) followed by click chemistry. For this, dTAG- or DMSO-treated cells were pulse-labelled with 1 mM EU (Jena Bioscience, CLK-N002-10) for 30 min. One million cells were collected and fixed with 4% formaldehyde and permeabilized with a saponin-based wash. Then, cells were incubated with a Click Cocktail (1 mM CuSO4, 43 mM Tris, 129 mM NaCl, 5.6 mM C₆H₇NaO₆, 50 M CalFluor 488 Azide (Click Chemistry Tools, 1369-1)) for 30 min at room temperature in the dark. After incubation, DNA was stained with FxCycle Violet Stain (Invitrogen, F10347) in FACS Buffer for 30 minutes at room temperature before analysis by flow cytometry. Cells treated with Actinomycin D (2 μM) for 2 h were used as a negative control. A surprisingly modest reduction in the rate of cellular RNA synthesis was detected, which remained high at 65% at the time of a complete proliferative arrest 4 days after dTAG addition (FIGS. 6A and 13A-13B). Indeed, of the monitored cellular functions, the overall rate of transcription was the least affected, still at 17% after day 7 (FIG. 13B).

Collectively, these results document a coherent decline in critical cellular functions that leads to quiescence in cells with very low levels of RNase MRP. The arrested DNA synthesis, minimal protein synthesis, and relatively higher metabolic and transcriptional activities suggest active maintenance of the quiescent state.

### Example 2

This example describes the use of a nonlimiting example pharmacological approach to induce cellular quiescence via inhibition of rRNA transcription by RNA polymerase I (Pol I) in different human and mouse cells. Aspects of these results were disclosed in the peer review file associated with Liu et al., Nat Commun 2025 (PMID: 40533478, which is incorporated herein in its entirety).

Modulation of cellular fate pharmacologically, through use of drugs, including but not limited to compounds of relatively small molecular weight, designed and synthesized or discovered through chemical means, or peptides or their derivatives, is an attractive approach that does not require prior manipulation of cells. A number of drugs, including peptides and small molecular inhibitors, have been proposed that inhibit ribosome biogenesis, especially transcription of 47S pre-rRNA by Pol I. Search for small molecule inhibitors of ribosome biogenesis has largely been motivated by realization that ribosome biogenesis is significantly enhanced in tumor cells, suggesting that these might be more sensitive to inhibition of ribogenesis compared to normal cells, thus presenting a potential opportunity to selectively target cancer cells. Indeed, several small molecule inhibitors of RNA polymerase I have been explored for treatment of cancer. One important realization emanating from relevant studies has been that inhibition of RNA polymerase I can selectively kill certain cancer cells, while maintaining viability of normal, wild-type cells. However, with much attention on cancer cells, the effect of Pol I inhibition on normal cells has not been thoroughly investigated.

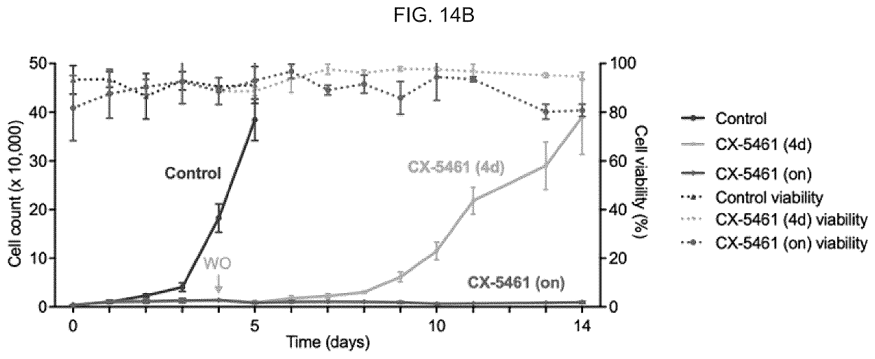

Several Pol I inhibitors differing in their potencies and specificities have been identified, including, for example, cisplatin, oxaliplatin, 5-fluorouracil, methotrexate, actinomycin D, doxorubicin, mitoxantrone, mitomycin C, campthotecin, irinotecan, etoposide, ellipticines, CX-3543, CX-5461, BMH-21, and PMR-116. Whereas several of these rather non-selectively inhibit Pol I, a handful of compounds, such as CX-5461 (FIG. 14A), were found to be more selective.

To investigate the effect of CX-5461 on cell growth, wild-type HEK293T cells, grown in DMEM supplemented with 10% FBS (Cytiva, SH30910.03) and 100 U/ml Penicillin-Streptomycin (Gibco, 15140122) at 37° C. and 5% CO₂, were treated with different concentrations of CX-5461 and monitored the effect on cell proliferation and viability over time. Cells were seeded at 5,000 cells per well in a 48-well plate in 1 ml of media and every 4 days, 200 μl of CX-5461 at the tested concentration was added. At each analyzed time point, cells were harvested by digestion with 0.25% trypsin-EDTA (Gibco, 2661762) from individual wells, and then counted using a hemocytometer (Fisher Scientific, 0267151B). Cell viability was determined via staining with Trypan Blue (Sigma, T8154-100 ml). To wash out CX-5461 from cultures, cell culture supernatant was removed, attached cells were harvested by digestion with 0.25% trypsin-EDTA, combined with the supernatant, and washed twice with complete media. Cells were then seeded into a new 48-well plate and counted at each indicated time point.

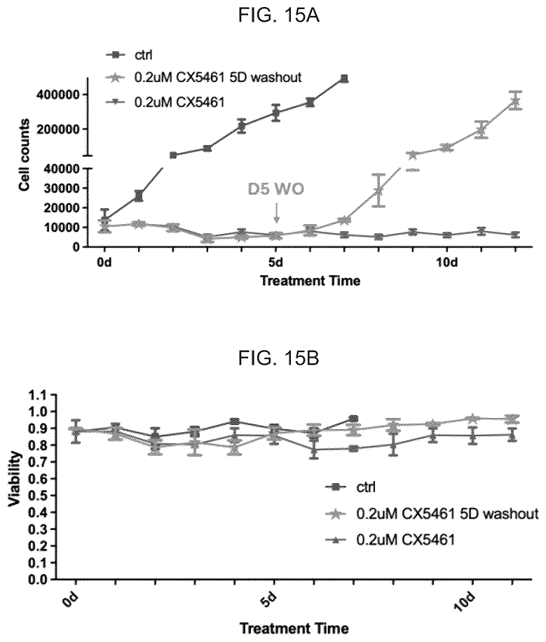

Treatment with 2 μM CX-5461 led to a complete arrest of cell proliferation at 2 to 3 days of incubation with no significant effect on cell viability, a state that could be maintained for at least four weeks in culture (FIG. 14B). Notably, removal of CX-5461 at 4 days of incubation allowed cells to resume proliferation within about 2 days (FIG. 14B). A similar test of the CX-5461 compound on mouse NIH/3T3 fibroblast cells, grown under the same conditions as HEK293T cells, found analogously rapid onset of proliferative arrest, within about 1 to 2 days, that could be released and cells allowed to re-enter proliferation by washing out CX-5461 (FIGS. 15A-15B).

These results demonstrate induction of cellular quiescence in cells of different species by pharmacological inhibition of ribosome biogenesis.

### Example 3

This example describes a nonlimiting example use of the auxin-inducible degron (AID) system in human cells for inhibition of Pol I via targeted degradation of one of its subunits and consequent induction of cellular quiescence.

The eukaryotic Pol I shows many similarities to Pol II, Pol III, and archaeal or even bekial systems, especially in RNA polymerase architecture and mechanism of catalysis.

The catalytic core of Pol I, which drives transcription of a single target gene, the 47S pre-rRNA, comprises of several subunits, including the Pol I-specific RPA1 (also known as RPA194), which is the largest subunit containing the active site responsible for RNA synthesis.

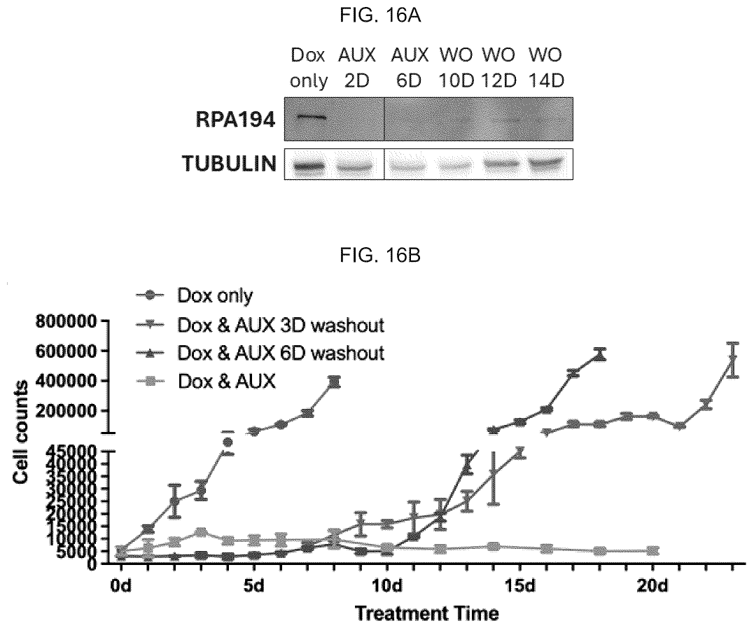

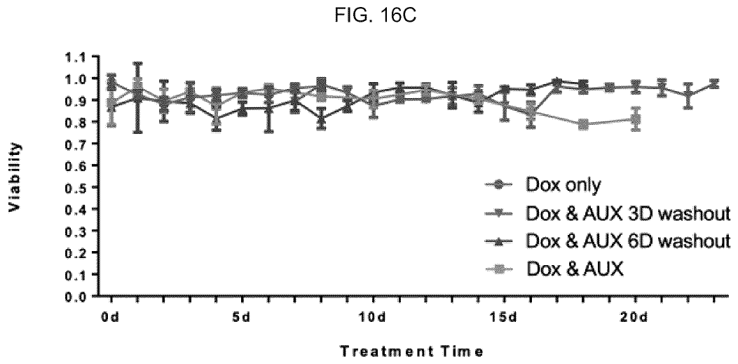

To further showcase the variety of technologies that can be used to induce cellular quiescence via inhibition of ribosome biogenesis, as well as to validate the specificity of the aforementioned pharmacological approaches (see Example 2), the AID system was used for targeted degradation of the RPA194 subunit of Pol I to induce cellular quiescence. Human HCT116 cells, grown and seeded as above, carrying a cassette encoding mini-AID homozygously at the initiation site of the endogenous RPA194 gene locus were treated with auxin, which led to depletion of the RPA194 protein (FIG. 16A). Importantly, akin to CX-5461 treated HEK293T or NIH/3T3 cells (FIGS. 14A-14B and 15A-15B), the auxin-treated AID degron-edited HCT116 cells stopped proliferating within about 2 days of treatment, remained viable, and readily re-entered the cell cycle upon removal of auxin (FIGS. 16B-16C). This demonstrates induction of cellular quiescence using the AID system for targeted inhibition of ribogenesis.

### Example 4

This example describes targeting of the LASIL endonuclease to inhibit ribosome biogenesis in human cells and consequent induction of cellular quiescence. This example also demonstrates the feasibility of a modified dTAG-based approach for induction of cellular quiescence.

Following transcription by Pol I, the 47S pre-rRNA is processed through a tightly coordinated cascade of endo- and exo-nucleolytic cleavages. The early-acting endonuclease RNase MRP splits pre-rRNA into segments destined for incorporation into the 40S or 60S ribosomal subunits. Further downstream cleavages are required to cut and trim the pre-rRNA into the 18S, 5.8S, and 28S rRNAs for incorporation into the ribosomal subunits (FIG. 1). One of the downstream enzymes is the LASIL endonuclease, which cuts in the ITS2 region of pre-rRNA to split the future 5.8S from the 28S rRNA (future 5.8S from the 25S rRNA in yeast), both of which become incorporated into the 60S subunit of the ribosome.

To investigate if targeting LASIL might, akin to inhibition of Pol I or RNase MRP, induce cellular quiescence, a modified dTAG-based approach was used, where in HEK293T cells, via lentiviral infection, ectopically expressed human LASIL fused in-frame with the KFBP12F36V degradation tag expressed from the pLEX_305-C-dTAG vector (Addgene, 91798). CRISPR/Cas9-assisted disruption of the endogenous LASIL locus using the px333 vector (Addgene, 64073) was then used, to homozygously introduce out-of-frame deletions to abolish expression of the endogenous LASIL protein. This allowed for expression exclusively of the ectopic, degradation tag-fused, but not endogenous LASIL protein.

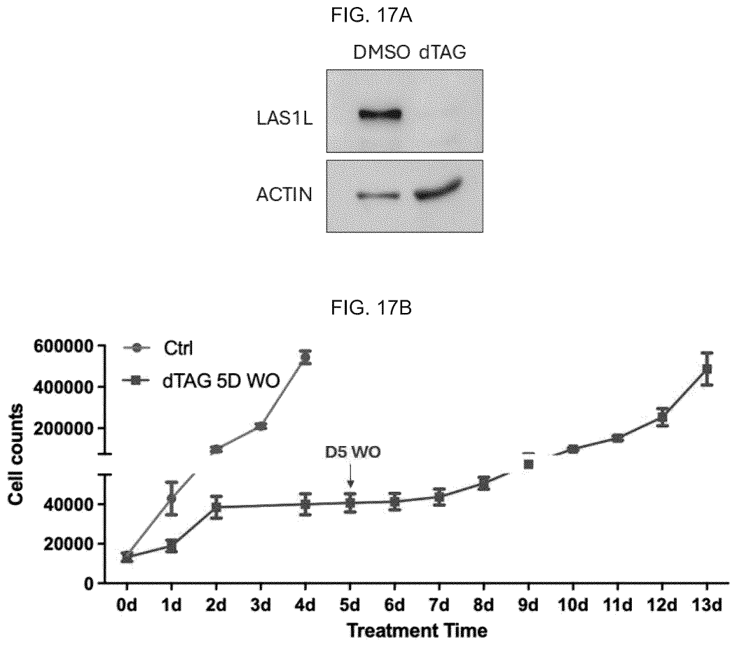

Treatment of the edited HEK293T cells with dTAG induced rapid degradation of the ectopic LASIL protein within 1 hour (FIG. 17A), which resulted in cellular quiescence whose dynamics resembled that seen in other mouse or human cells induced to quiescence by targeting ribosome biogenesis via pharmacological or chemical biology-based approaches (FIGS. 6A, 14B, 15A, and 16B).

This disclosure is susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method of inducing quiescence in a cell, tissue, organ, organoid, organ system, tumor, organism, or any combination thereof, the method comprising:
administering to the cell, tissue, organ, organoid, tumor, organ system, or organism an effective concentration of a quiescence-inducing agent,
wherein the effective concentration of the quiescence-inducing agent is one in which the total number of active ribosomes is reduced in the cell, tissue, organ, organoid, tumor, organ system, or organism compared to an untreated cell, tissue, organ, organoid, tumor, or organism.

2. The method of claim 1, wherein the quiescence-inducing agent inhibits ribosomal and/or rRNA biogenesis.

3. The method of claim 1, wherein the quiescence-inducing agent inhibits activity, expression, and/or the total number of an at least one ribosome biogenesis protein and/or an at least one rRNA biogenesis protein present in a cell, tissue, organ, organoid, organ system, tumor, and/or organism.

4. The method of claim 3, wherein the at least one ribosome biogenesis protein and/or the at least one rRNA biogenesis protein is involved in at least one of: rRNA transcription, rRNA processing, rRNA modification, ribosomal protein formation, transport of assembled ribosomal subunits, or any combination thereof.

5. The method of claim 1, wherein the quiescence-inducing agent inhibits activity and/or reduces the total copy number of RNA polymerase I, RNase MRP, LASIL, or any combination thereof.

6. The method of claim 5, wherein the quiescence-inducing agent is CX-5461 or a derivative thereof.

7. The method of claim 3, wherein the quiescence-inducing agent induces targeted degradation of the ribosome biogenesis protein and/or rRNA biogenesis protein.

8. The method of claim 7, wherein the quiescence-inducing agent induces targeted degradation through a degradation tag, and wherein the degradation tag is FKBP12^{F36V}.

9. The method of claim 1, wherein the quiescence-inducing agent comprises an auxin-inducible degron system and auxin or a derivative thereof.

10. The method of claim 1, wherein the quiescence-inducing agent inhibits expression of a gene involved in ribosome biogenesis via RNA interference, an antisense oligonucleotide, or a CRISPR-based method.

11. The method of claim 1, wherein total number of active ribosomes in the cell, tissue, organ, organoid, tumor, or organism is reduced by at least about 10%, 20%, 30%, 40%, 50%, 60%, 70%, 80%, 90%, 95%, 99%, 100%, or any number between about 10% and about 100%, compared to an analogous cell, tissue, organ, organoid, tumor, or organism that is not administered the quiescence-inducing agent.

12. The method of claim 1, the method further comprising lowering the temperature of the plurality of cells to below 35° C., freezing, or vitrifying the plurality of cells during or after induction of quiescence.

13. The method of claim 1, wherein the quiescence-inducing agent is administered in vitro, ex vivo, or in situ.

14. The method of claim 1, wherein the quiescent state is maintained for a period of at least about 2, 4, 6, 8, 10, 12, or 14 days.

15. The method of claim 1, the method further comprising inhibiting and/or removing the quiescence-inducing agent, wherein the cell, tissue, organ, organoid, tumor, or organism is released from quiescence.

16. The method of claim 15, wherein inhibiting and/or removing the quiescence-inducing agent comprises washing the cells and/or omitting further administration of the quiescence-inducing agent.

17. The method of claim 15, further comprising contacting the cell, tissue, organ, organoid, tumor, or organism with a nutrient-enriched medium to promote recovery from quiescence.

18. A method of reducing metabolic activity in a non-dividing cell, tissue, organ, organoid, tumor, or organism, the method comprising administering a quiescence-inducing agent, thereby decreasing energy consumption while maintaining viability.

19. A method of promoting the viability and/or shelf-life of a cell, tissue, organ, organoid, tumor, or organism, the method comprising administering a quiescence-inducing agent.

20. The method of claim 19, further comprising cryopreserving the cell, tissue, organ, organoid, tumor, organ system, or organism.
